(19) United States
(12) Patent Application Publication
(10) Pub. No.: US 2005/0009803 A1
(43) Pub. Date: Jan. 13, 2005

(54) MESO-oxygenated texaphyrin analogues

(75) Inventors: Lei Fu, Fremont, CA (US); Tarak D. Mody, Sunnyvale, CA (US); Zhong Wang, Sunnyvale, CA (US)

Correspondence Address: Vinit G. Kathardekar;Pharmacyclics, Inc., 995 E. Arques Avenue, Sunnyvale, CA, 94085, US

(73) Assignee: Pharmacyclics, Inc., Sunnyvale, CA (US)

(21) Appl. No.: 10/911,284

(22) Filed: Aug. 4, 2004

Related U.S. Application Data
(63) Continuation-in-part of application No. 10/310,592, filed on Dec. 4, 2002, now abandoned.

(51) Int. Cl. A61K 31/555; C07F 5/00

(52) U.S. Cl. 514/185; 534/15; 540/465

(57) ABSTRACT

The present invention provides compounds of Formula I 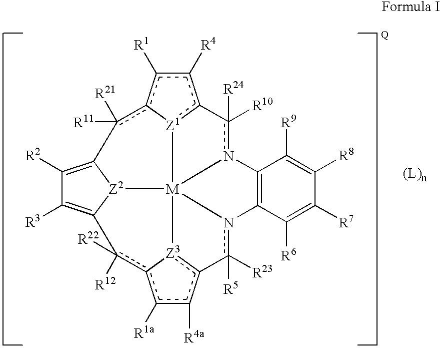 its pharmaceutically acceptable salts, hydrate and prodrug forms thereof.

### CLAIM OF PRIORITY

This application is a continuation-in-part of U.S. patent application Ser. No. 10/310,592, filed Dec. 4, 2002 (Attorney Docket No. 4236.00 US), the content of which is incorporated herein by reference in its entirety.

### FIELD OF INVENTION

The present invention relates to novel compounds of Formula I, processes to prepare these compounds and methods of using these compounds.

### BACKGROUND OF THE INVENTION

Texaphyrins have been described as aromatic pentadentate benzannulene compounds containing both 18π- and 22π-electron delocalization pathways, which have the ability to integrate metals within their core to form complexes known as “metallotexaphyrins.” While a variety of metals have been described in forming metallotexaphyrins, the preferred metals have been the lanthamides (and lanthanoids, such as y³⁺), most notably Gd³⁺ and Lu³⁺. Texaphyrins and metallotexaphyrins have been described, among other things, as chemosensitizers in both cancer and arteriosclerosis treatment, and as photosensitizers in photodynamic therapy of cancer, atherosclerosis, and ophthalmology.

Notwithstanding such prior teachings, the oxo or hydroxy texaphyrins have remained neither taught nor suggested in the relevant literature. In light of the recent discoveries related to the redox chemistry of metallotexaphyrins, the present inventors have identified the advantage of providing such oxo or hydroxy texaphyrins as supporting complexation with metal cations having higher oxidation states than those previously employed.

### SUMMARY OF THE INVENTION

The present invention provides compounds of Formula I 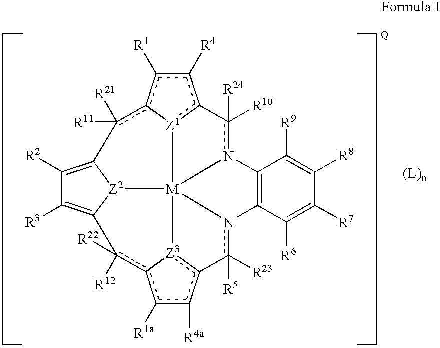
its hydrate, pharmaceutically acceptable salt or prodrug form thereof, wherein:
M represents H or a metal cation;
Q represents an integer of from about −5 to about +5;
L represents a charge balancing species;
“n” represents an integer of from 0 to +5;
Z¹, Z² and Z³ independently represent N, O, CH or S;
R¹, R^{1a}, R², R³, R⁴, R^{4a}, R⁷, and R⁸ are independently selected from acyl, acyloxy, optionally substituted alkenyl, optionally substituted alkoxy, optionally substituted alkyl, optionally substituted alkynyl, optionally substituted amino, optionally substituted aryl, optionally substituted aryloxy, carboxyl, (optionally substituted alkoxy)carbonyl, (optionally substituted amino)carbonyl, (optionally substituted alkoxy)carbonyloxy, (optionally substituted amino)carbonyloxy, cyano, optionally substituted cycloalkyl, optionally substituted cycloalkenyl, halogen, optionally substituted heteroaryl, optionally substituted heteroaryloxy, optionally substituted heterocyclyl, optionally substituted heterocyclooxy, hydrogen, hydroxyl, nitro, optionally substituted azo, S—R³¹, SO—R³¹, SO₂—R³¹, and the moiety X-Y;
R⁶ and R⁹ are independently selected from acyl, acyloxy, optionally substituted alkenyl, optionally substituted alkoxy, optionally substituted alkyl, optionally substituted alkynyl, optionally substituted amino, optionally substituted aryl, optionally substituted aryloxy, carboxyl, (optionally substituted alkoxy)carbonyl, (optionally substituted amino)carbonyl, (optionally substituted alkoxy)carbonyloxy, (optionally substituted amino)carbonyloxy, cyano, optionally substituted cycloalkyl, optionally substituted cycloalkenyl, fluoro, chloro, bromo, optionally substituted heteroaryl, optionally substituted heteroaryloxy, optionally substituted heterocyclyl, optionally substituted heterocyclooxy, hydrogen, hydroxyl, nitro, optionally substituted azo, sulfanyl, sulfinyl, sulfonyl, and the moiety X-Y;
R⁵, R¹⁰, R¹¹ and R¹² are independently selected from acyl, optionally substituted alkoxy, optionally substituted alkyl, optionally substituted aryl, halo, hydrogen, hydroxy, optionally substituted cycloalkyl, optionally substituted cycloalkenyl, optionally substituted heteroaryl, and optionally substituted heterocyclyl;
R²¹, R²², R²³ and R²⁴ are optional substituents, and when present are independently selected from H, OH, optionally substituted C₁₋₄ alkyl, optionally substituted C₃₋₁₀ cycloalkyl, optionally substituted aryl, optionally substituted heteroaryl, optionally substituted heterocyclyl;
X is a covalent bond or a linker;
Y is a catalytic group, a chemotherapeutic agent or a site-directing group;
R³¹ represents acyl, optionally substituted alkenyl, optionally substituted alky, optionally substituted alkoxy, optionally substituted alkoxycarbonyl, optionally substituted alkynyl, optionally substituted aminocarbonyl, optionally substituted aryl, carboxy, optionally substituted cycloalkyl, optionally substituted heteroaryl, or optionally substituted heterocyclyl; alternatively
R⁵ and R²³ along with the carbon atom to which they are attached represent C═O, C═S, C═CHR³¹ or C═N—R³¹;
R¹⁰ and R²⁴ along with the carbon atom to which they are attached represent C═O, C═S, C═CHR³¹ or C═N—R³¹;
R¹¹ and R²¹ along with the carbon atom to which they are attached represent C═O, C═S, C═CHR³¹ or C═N—R³¹;
R¹² and R²² along with the carbon atom to which they are attached represent C═O, C═S, C═CHR³¹ or C═N—R³¹;
R¹ and R²¹,
R¹¹ and R²,
R³ and R²²,
R¹² and R^{1a},
R^{4a} and R⁵,
R²³ and R⁶,
R⁹ and R²⁴, and
R¹⁰ and R⁴ respectively taken together represent W(CHR³²)₁₋₈ or (CHR³²)₁₋₈, wherein R³² represents H, methyl, or ethyl, and W represents O, NR³¹, or S, with the proviso that:
(a) at least one of
   R⁵ and R²³,
   R¹⁰ and R²⁴,
   R¹¹ and R²¹, and
   R¹² and R²² respectively taken together with the carbon atom to which they are attached represent C═O, C═S, C═CHR³¹ or C═N—R³¹; or
(b) at least one of
   R¹ and R²¹,
   R¹¹ and R²,
   R³ and R²²,
   R¹² and R^{1a},
   R^{4a} and R⁵,
   R²³ and R⁶,
   R⁹ and R²⁴, and
   R¹⁰ and R⁴ respectively, taken together represent W(CHR³²)₁₋₈ or (CHR³²)₁₋₈, where R³² represents H, methyl or ethyl, and represents O, NR³¹, or S.

### DETAILED DESCRIPTION

In one aspect of the present invention are provided compounds of Formula I, 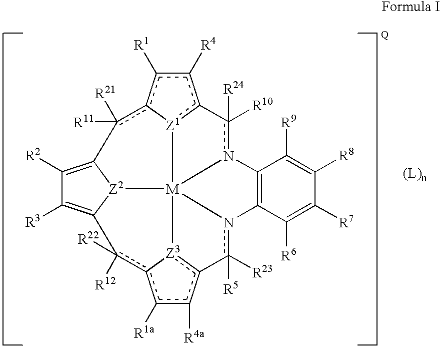
its hydrate, pharmaceutically acceptable salt or prodrug form thereof, wherein:
M represents H or a metal cation;
Q represents an integer of from −5 to +5;
L represents a charge balancing species;
“n” represents an integer of from 0 to +5;
Z¹, Z² and Z³ independently represent N, O, CH₂ or S;
R¹, R^{1a}, R², R³, R⁴, R^{4a}, R⁷, and R⁸ are independently selected from acyl, acyloxy, optionally substituted alkenyl, optionally substituted alkoxy, optionally substituted alkyl, optionally substituted alkynyl, optionally substituted amino, optionally substituted aryl, optionally substituted aryloxy, carboxyl, (optionally substituted alkoxy)carbonyl, (optionally substituted amino)carbonyl, (optionally substituted alkoxy)carbonyloxy, (optionally substituted amino)carbonyloxy, cyano, optionally substituted cycloalkyl, optionally substituted cycloalkenyl, halogen, optionally substituted heteroaryl, optionally substituted heteroaryloxy, optionally substituted heterocyclyl, optionally substituted heterocyclooxy, hydrogen, hydroxyl, nitro, optionally substituted azo, S—R³¹, SO—R³¹, SO₂—R³¹, and the moiety X-Y;
R⁶ and R⁹ are independently selected from acyl, acyloxy, optionally substituted alkenyl, optionally substituted alkoxy, optionally substituted alkyl, optionally substituted alkynyl, optionally substituted amino, optionally substituted aryl, optionally substituted aryloxy, carboxyl, (optionally substituted alkoxy)carbonyl, (optionally substituted amino)carbonyl, (optionally substituted alkoxy)carbonyloxy, (optionally substituted amino)carbonyloxy, cyano, optionally substituted cycloalkyl, optionally substituted cycloalkenyl, fluoro, chloro, bromo, optionally substituted heteroaryl, optionally substituted heteroaryloxy, optionally substituted heterocyclyl, optionally substituted heterocyclooxy, hydrogen, hydroxyl, nitro, optionally substituted azo, sulfanyl, sulfinyl, sulfonyl, and the moiety X-Y;
R⁵, R¹⁰, R¹¹ and R¹² are independently selected from acyl, optionally substituted alkoxy, optionally substituted alkyl, optionally substituted aryl, halo, hydrogen, hydroxy optionally substituted cycloalkyl, optionally substituted cycloalkenyl, optionally substituted heteroaryl, and optionally substituted heterocyclyl;
R²¹, R²², R²³ and R²⁴ are optional substituents, and when present are independently selected from H, OH, optionally substituted C₁₋₄ alkyl, optionally substituted C₃₋₁₀ alkyl, optionally substituted aryl, optionally substituted heteroaryl, optionally substituted heterocyclyl;
X is a covalent bond or a linker;
Y is a catalytic group, a chemotherapeutic agent or a site-directing group; alternatively
R⁵ and R²³ along with the carbon atom to which they are attached represent C═O, C═S, C═CHR³¹ or C═N—R³¹;
R¹⁰ and R²⁴ along with the carbon atom to which they are attached represent C═O, C═S, C═CHR³¹ or C═N—R³¹;
R¹¹ and R²¹ along with the carbon atom to which they are attached represent C═O, C═S, C═CHR³¹ or C═N—R³¹;
R¹² and R²² along with the carbon atom to which they are attached represent C═O, C═S, C═CHR³¹ or C═N—R³¹;
R³¹ represents acyl, optionally substituted alkenyl, optionally substituted alky, optionally substituted alkoxy, optionally substituted alkoxycarbonyl, optionally substituted alkynyl, optionally substituted aminocarbonyl, optionally substituted aryl, carboxy, optionally substituted cycloalkyl, optionally substituted heteroaryl, or optionally substituted heterocyclyl;
with the proviso that:
   at least one of
      R⁵ and R²³,
      R¹⁰ and R²⁴,
      R¹¹ and R²¹, and
      R¹² and R²²,
         along with the respective carbon atoms to which they are attached represent C═O, C═S, C═CHR³¹ or C═N—R³¹.

A preferred embodiment provides a compound of Formula I wherein,
M is selected from H, and a divalent or trivalent metal cation;
Z¹ and Z² independently represent N, O or S;
Z³ represents N, O, CH₂ or S;
R¹, R^{1a}, R², R³, R⁴, R^{4a}, R⁷, and R⁸ are independently selected from acyl, acyloxy, optionally substituted alkoxy, optionally substituted alkyl, optionally substituted amino, optionally substituted aryl, optionally substituted aryloxy, carboxyl, (optionally substituted amino)carbonyl, cyano, optionally substituted cycloalkyl, optionally substituted cycloalkenyl, halogen, optionally substituted heteroaryl, optionally substituted heteroaryloxy, optionally substituted heterocyclyl, hydrogen, hydroxyl, nitro, optionally substituted azo, S—R³¹, SO—R³¹, and SO₂—R³¹;
R⁶ and R⁹ are independently selected from acyl, acyloxy, optionally substituted alkoxy, optionally substituted alkyl, optionally substituted amino, optionally substituted aryl, optionally substituted aryloxy, carboxyl, (optionally substituted amino)carbonyl, cyano, optionally substituted cycloalkyl, optionally substituted cycloalkenyl, halogen, optionally substituted heteroaryl, optionally substituted heteroaryloxy, optionally substituted heterocyclyl, hydrogen, hydroxyl, nitro, optionally substituted azo, S—R³¹, SO—R³¹, and SO₂—R³¹;
R²¹, R²², R²³ and R²⁴ are optional substituents, and when present are independently selected from H, optionally substituted C₁₋₄ alkyl, optionally substituted C₃₋₁₀ cyclo alkyl, optionally substituted aryl, optionally substituted heteroaryl, optionally substituted heterocyclyl; alternatively
R⁵ and R²³ along with the carbon atom to which they are attached represent C═O, C═S, or C═N—R³¹;
R¹⁰ and R²⁴ along with the carbon atom to which they are attached represent C═O, C═S, or C═N—R³¹;
R¹¹ and R²¹ along with the carbon atom to which they are attached represent C═O, C═S, or C═N—R³¹;
R¹² and R²² along with the carbon atom to which they are attached represent C═O, C═S, or C═N—R³¹;
R³¹ represents acyl, optionally substituted alky, optionally substituted alkoxy, optionally substituted aminocarbonyl, optionally substituted aryl, carboxy, optionally substituted cycloalkyl, optionally substituted heteroaryl, or optionally substituted heterocyclyl;
with the proviso that at least one of
   R⁵ and R²³,
   R¹⁰ and R²⁴,
   R¹¹ and R²¹ and
   R¹² and R²² along with the respective carbon atoms to which they are attached represent C═O or C═N—R³¹.

A further preferred embodiment provides a compound of Formula I, wherein
Z³ represents N, O, or S;
M represents H or a metal cation selected from Lu(III), Fe(II), Fe(III), Ni(II), Cd(II), In(III), Gd (III), Sm(III), Eu(III), Tb(III), Dy(III), Er(III), Tm(III), Yb(II), Mn(II), Y(III), and Co (II); and L represents a charged species selected from H⁺, NH₄⁺, Na⁺, Ca²⁺, K⁺, Ba²⁺, Cl⁻, Br⁻, SO₄²⁻, carboxylate (CH₃COO⁻, C₂H₅COO⁻), sulfonate (RSO₃⁻), phosphate, cholate, gluconate, glucuronate and citrate.

Another preferred embodiment provides a compound of Formula I wherein,
M represents Lu(III), Gd (III), Er(III), Tb(III), Dy(III), Ho(III), Y(III), Tm(III), Yb(III), or Mn(II); Z¹, Z² and Z³ are independently selected from N and O;
R¹, R^{1a}, R², R³, R⁴, R^{4a}, R⁷, and R⁸ are independently selected from C₁₋₅ alkyl, C₁₋₅ hydroxyalkyl, C₁₋₅ alkoxyalkyl, C₁₋₅ carboxyalkyl, acetoxyalkyl, aryloxyalkyl, R(CH₂CH₂O)ₙ, where n=1-5, and R═C₁₋₂ alkyl, phenyl, hydroxyl, carboxyl;
R⁶ and R⁹ are independently selected from acyl, acyloxy, optionally substituted alkoxy, optionally substituted alkyl, optionally substituted amino, optionally substituted aryl, optionally substituted aryloxy, carboxyl, (optionally substituted alkoxy)carbonyl, (optionally substituted amino)carbonyl, (optionally substituted amino)carbonyloxy, cyano, fluoro, chloro, hydrogen, hydroxyl, and nitro;
R⁵, R¹⁰, R¹¹ and R¹² are independently selected from H, OH, C₁₋₂ alkyl, C₁₋₃ acyl, and phenyl; and
R²¹, R²², R²³ and R²⁴ are optional substituents, and when present are independently selected from H, OH, optionally substituted C₁₋₄ alkyl, optionally substituted C₃₋₁₀ alkyl, optionally substituted aryl, optionally substituted heteroaryl, optionally substituted heterocyclyl; alternatively
R⁵ and R²³ along with the carbon atom to which they are attached represent C═O or C═N—R³¹;
R¹⁰ and R²⁴ along with the carbon atom to which they are attached represent C═O or C═N—R³¹;
R¹¹ and R²¹ along with the carbon atom to which they are attached represent C═O or C═N—R³¹;
R¹² and R²² along with the carbon atom to which they are attached represent C═O or C═N—R³¹;
R³¹ represents acyl, optionally substituted alky, optionally substituted alkoxy, optionally substituted alkoxycarbonyl, optionally substituted aminocarbonyl, optionally substituted aryl, or carboxy;
with the proviso that at least one of
   R⁵ and R²³,
   R¹⁰ and R²⁴,
   R¹¹ and R²¹, and
   R¹² and R²² along with the respective carbon atoms to which they are attached represent C═O.

A particularly preferred embodiment provides a compound of Formula I wherein,
M represents Lu(III), Gd(III), Tb(III), Mn(II), Co(II), Y(III), or Ho(III); and
Z¹, Z² and Z³ independently represent N.

Also provided by the present invention is a compound of Formula I: 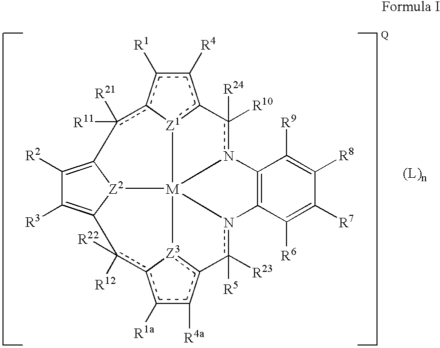
its hydrate, pharmaceutically acceptable salt, or prodrug form thereof, wherein:
M represents Lu(III), Gd(III), Tb(III), Mn(II), Co(II), Y(III), or Ho(III);
Z¹, Z² and Z³ independently represent N or O;
R¹, R^{1a}, R², R³, R⁴, R^{4a}, R⁷, and R⁸ are independently selected from C₁₋₅ alkyl, C₁₋₅ hydroxyalkyl, C₁₋₅ alkoxyalkyl, C₁₋₅ carboxyalkyl, acetoxyalkyl, aryloxyalkyl, R(CH₂CH₂O)ₙ, where n=1-5, and R═C₁₋₂ alkyl, phenyl, hydroxyl, carboxyl;
R⁶ and R⁹ are independently selected from acyl, acyloxy, optionally substituted alkoxy, optionally substituted alkyl, optionally substituted amino, optionally substituted aryl, optionally substituted aryloxy, carboxyl, (optionally substituted alkoxy)carbonyl, (optionally substituted amino)carbonyl, (optionally substituted amino)carbonyloxy, cyano, fluoro, chloro, hydrogen, hydroxyl, and nitro;
R⁵, R¹⁰, R¹¹ and R¹² are independently selected from H, OH, C₁₋₂ alkyl, C₁₋₃ acyl, and phenyl;
R²¹, R²², R²³ and R²⁴ are optional substituents, and when present are independently selected from H, OH, optionally substituted C₁₋₄ alkyl, optionally substituted C₃₋₁₀ alkyl, optionally substituted aryl, optionally substituted heteroaryl, optionally substituted heterocyclyl; alternatively
R⁵ and R²³ along with the carbon atom to which they are attached represent C═O or C═N—R³¹;
R¹⁰ and R²⁴ along with the carbon atom to which they are attached represent C═O or C═N—R³¹′;
R¹¹ and R²¹ along with the carbon atom to which they are attached represent C═O or C═N—R³¹;
R¹² and R²² along with the carbon atom to which they are attached represent C═O or C═N—R³¹;
R³¹ represents acyl, optionally substituted alky, optionally substituted alkoxy, optionally substituted alkoxycarbonyl, optionally substituted aminocarbonyl, optionally substituted aryl, or carboxy;
with the proviso that at least one of
   R⁵ and R²³,
   R¹⁰ and R²⁴,
   R¹¹ and R²¹, and
   R¹² and R²² along with the respective carbon atoms to which they are attached represent C═O. A further preferred embodiment of this invention provides a compound of Formula I: 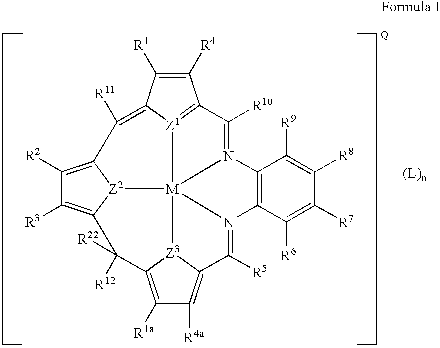
      its hydrate, pharmaceutically acceptable salt, or prodrug form thereof, wherein:
M represents Lu(III) or Gd(III);
L represents OAc;
Z¹, Z² and Z³ represent N;
R¹ and R^{1a} represent (CH₂)₃OH;
R² represents C₂H₅;
R³ represents C₂H₅;
R⁴ and R^{4a} CH₃;
R⁵ represents H;
R⁶ represents CH₃;
R⁷ and R⁸ are independently selected from H and O—(CH₂—CH₂—O)₃—CH₃;
R⁹ represents CH₃;
R¹⁰ and R¹¹ represent H; and
R¹² and R²² along with the carbon atom to which they are attached represent C═O.

Provided in yet another aspect of the present invention is a compound of Formula I 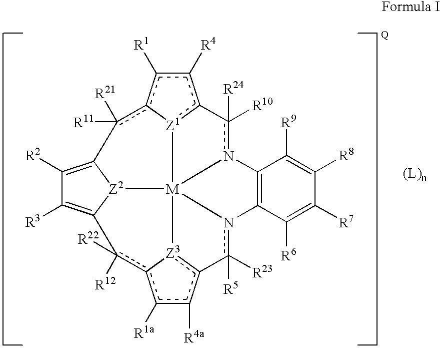
its hydrate, pharmaceutically acceptable salt or prodrug form thereof, wherein:
M represents H or a metal cation;
Q represents an integer of from −4 to +4;
L represents a charge-balancing group;
“n” represents an integer of from about 0 to about +4;
Z¹, Z² and Z³ independently represent N, O, CH or S;
R¹, R^{1a}, R², R³, R⁴, R^{4a}, R⁷ and R⁸ are independently selected from acyl, acyloxy, optionally substituted alkenyl, optionally substituted alkoxy, optionally substituted alkyl, optionally substituted alkynyl, optionally substituted amino, optionally substituted aryl, optionally substituted aryloxy, carboxyl, (optionally substituted alkoxy)carbonyl, (optionally substituted amino)carbonyl, (optionally substituted alkoxy)carbonyloxy, (optionally substituted amino)carbonyloxy, cyano, optionally substituted cycloalkyl, optionally substituted cycloalkenyl, halogen, optionally substituted heteroaryl, optionally substituted heteroaryloxy, optionally substituted heterocyclyl, optionally substituted heterocyclooxy, hydrogen, hydroxyl, nitro, optionally substituted azo, sulfanyl, sulfinyl, sulfonyl, and the moiety X-Y;
R⁶ and R⁹ are independently selected from acyl, acyloxy, optionally substituted alkenyl, optionally substituted alkoxy, optionally substituted alkyl, optionally substituted alkynyl, optionally substituted amino, optionally substituted aryl, optionally substituted aryloxy, carboxyl, (optionally substituted alkoxy)carbonyl, (optionally substituted amino)carbonyl, (optionally substituted alkoxy)carbonyloxy, (optionally substituted amino)carbonyloxy, cyano, optionally substituted cycloalkyl, optionally substituted cycloalkenyl, fluoro, chloro, bromo, optionally substituted heteroaryl, optionally substituted heteroaryloxy, optionally substituted heterocyclyl, optionally substituted heterocyclooxy, hydrogen, hydroxyl, nitro, optionally substituted azo, sulfanyl, sulfinyl, sulfonyl, and the moiety X-Y;
R⁵, R¹⁰, R¹¹ and R¹² are independently selected from acyl, optionally substituted alkoxy, optionally substituted alkyl, optionally substituted aryl, halo, hydrogen, hydroxy optionally substituted cycloalkyl, optionally substituted cycloalkenyl, optionally substituted heteroaryl, optionally substituted heterocyclyl, and OH;
R²¹, R²², R²³ and R²⁴ are optional substituents, and when present are independently selected from H, OH, C₁₋₄ alkyl, C₃₋₁₀ cycloalkyl, aryl, heteroaryl, and heterocyclyl;
alternatively
   R¹ and R²¹,
   R¹¹ and R²,
   R³ and R²²,
   R¹² and R^{1a},
   R^{4a} and R⁵,
   R²³ and R⁶,
   R⁹ and R²⁴, and
   R¹⁰ and R⁴ respectively, taken together represent W(CHR³²)₁₋₈ or (CHR³²)₁₋₈, wherein R³² represents H, methyl, or ethyl, and W represents O, NR³¹, or S,
with the proviso that at least one of
   R¹ and R²¹,
   R¹¹ and R²,
   R³ and R²²,
   R¹² and R^{1a},
   R^{4a} and R⁵,
   R²³ and R⁶,
   R⁹ and R²⁴, and
   R¹⁰ and R⁴ respectively taken together represent W(CHR³²)₁₋₈ or (CHR³²)₁₋₈.

A preferred embodiment provides a compound wherein, M is selected from H, a divalent metal cation and a trivalent metal cation;
R¹, R^{1a}, R², R³, R⁴, R^{4a}, R⁷, and R⁸ are independently selected from acyl, acyloxy, optionally substituted alkoxy, optionally substituted alkyl, optionally substituted amino, optionally substituted aryl, optionally substituted aryloxy, carboxyl, cycloalkyl, optionally substituted cycloalkenyl, halogen, optionally substituted heteroaryl, optionally substituted heteroaryloxy, optionally substituted heterocyclyl, hydrogen, hydroxyl, nitro, optionally substituted azo, S—R³¹, SO—R³¹, and SO₂—R³¹;
R⁶ and R⁹ are independently selected from acyl, acyloxy, optionally substituted alkoxy, optionally substituted alkyl, optionally substituted amino, optionally substituted aryl, optionally substituted aryloxy, carboxyl, cycloalkyl, cycloalkenyl, halogen, optionally substituted heteroaryl, optionally substituted heteroaryloxy, optionally substituted heterocyclyl, hydrogen, hydroxyl, nitro, optionally substituted azo, S—R³¹, and SO₂—R³¹;
R²¹, R²², R²³ and R²⁴ are optional substituents, and when present are independently selected from H, C₁₋₄ alkyl, C₃₋₁₀ cyclo alkyl, optionally substituted aryl, five to ten membered heteroaryl, optionally substituted heterocyclyl; C₃₋₁₀ cyclo alkyl substituted with halogen, O(CH₂)₀₋₂—CH₃, C₁₋₄ alkyl substituted with halogen, O(CH₂)₀₋₂—CH₃, and five to ten membered heteroaryl substituted with halogen, C₁₋₄ alkyl, O(CH₂)₀₋₂—CH₃; alternatively
R³¹ represents acyl, optionally substituted alky, optionally substituted alkoxy, optionally substituted aminocarbonyl, optionally substituted aryl, carboxy, optionally substituted cycloalkyl, optionally substituted heteroaryl, or optionally substituted heterocyclyl;
alternatively, one or more of
   R¹ and R²¹,
   R¹¹ and R²,
   R³ and R²²,
   R¹² and R^{1a},
   R^{4a} and R⁵,
   R²³ and R⁶,
   R⁹ and R²⁴, and
   R¹⁰ and R⁴ respectively taken together represent O(CHR³¹)₁₋₄ or (CHR³¹)₁₋₄, wherein R³¹ represents H, methyl, or ethyl;
with the proviso that at least one of
   R¹ and R²¹,
   R¹¹ and R²,
   R³ and R²²,
   R¹² and R^{1a},
   R^{4a} and R⁵,
   R²³ and R⁶,
   R⁹ and R²⁴, and
   R¹⁰ and R⁴ respectively taken together represent O(CHR³²)₁₋₄ or (CHR³²)₁₋₄, wherein R³² represents H, methyl, or ethyl.

A preferred embodiment provides a compound of Formula I wherein, M represents H or a metal cation selected from Lu(III), Fe(II), Fe(III), Ni(II), Cd(II), In(III), Gd (III), Sm(III), Eu(III), Tb(III), Dy(III), Er(III), Tm(III), Yb(III), Mn(II), Y(III), and Co (II), the preferred metal cations being Lu(III), Gd (III), Er(II), Tb(III), Dy(III), Ho(III), Y(III), Tm(III), Yb(III), or Mn(II); and L represents a charged species selected from H⁺, NH₄₊, Na⁺, Ca²⁺, K⁺, Ba²⁺, Cl⁻, Br⁻, SO₄²⁻, carboxylate (CH₃COO⁻, C₂H₅COO⁻), sulfonate (RSO₃⁻), phosphate, cholate, gluconate, glucuronate, and citrate.

A further preferred embodiment provides a compound of Formula I wherein,
Z¹, Z² and Z³ are independently selected from N and O;
R¹, R^{1a}, R², R³, R⁴, R^{4a}, R⁷, and R⁸ are independently selected from C₁₋₅ alkyl, C₁₋₅ hydroxyalkyl, C₁₋₅ alkoxyalkyl, C₁₋₅ carboxyalkyl, acetoxyalkyl, aryloxyalkyl, R(CH₂CH₂O)ₙ, where n=1-5, and R═C₁₋₂ alkyl, phenyl, hydroxyl, carboxyl;
R⁶ and R⁹ are independently selected from acyl, acyloxy, optionally substituted alkoxy, optionally substituted alkyl, optionally substituted amino, optionally substituted aryl, optionally substituted aryloxy, carboxyl, (optionally substituted alkoxy)carbonyl, (optionally substituted amino)carbonyl, (optionally substituted amino)carbonyloxy, cyano, fluoro, chloro, hydrogen, hydroxyl, and nitro;
R⁵, R¹⁰, R¹¹ and R¹² are independently selected from H, OH, C₁₋₂ alkyl, C₁₋₃ acyl, and phenyl;
R²¹, R²², R²³ and R²⁴ are optional substituents, and when present are independently selected from H, OH, optionally substituted C₁₋₄ alkyl, optionally substituted C₃₋₁₀ alkyl, optionally substituted aryl, optionally substituted heteroaryl, optionally substituted heterocyclyl;
alternatively, one or more of
   R¹ and R²¹,
   R¹¹ and R²,
   R³ and R²²,
   R¹² and R^{1a},
   R^{4a} and R⁵,
   R²³ and R⁶,
   R⁹ and R²⁴, and
   R¹⁰ and R⁴ respectively taken together represent O(CHR³²)₁₋₄ or (CHR³²)₁₋₄, wherein R³² represents H, methyl, or ethyl; with the proviso that at least one of
   R¹ and R²¹,
   R¹¹ and R²,
   R³ and R²²,
   R¹² and R^{1a},
   R^{4a} and R⁵,
   R²³ and R⁶,
   R⁹ and R²⁴, and
   R¹⁰ and R⁴ respectively taken together represent O(CHR³²)₂₋₄ or (CHR³²)₂₋₄, wherein R³² represents H, methyl, or ethyl.

Particularly preferred metal cation represented by M is selected from Lu(III), Gd(III), Tb(III), Mn(II), Co(II), Y(III), or Ho(III); and

Z¹, Z² and Z³ independently represent N.

Also provided by the present invention are compounds of Formula I 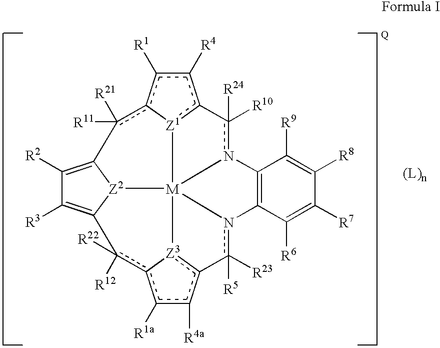
its hydrate, pharmaceutically acceptable salt or prodrug form thereof, wherein:
M represents Lu(III), Gd(III), Tb(III), Mn(II), Co(II), Y(III), or Ho(III);
Z¹, Z² and Z³ independently represent N;
R¹, R^{1a}, R², R³, R⁴, R^{4a}, R⁷, and R⁸ are independently selected from C₁₋₅ alkyl, C₁₋₅ hydroxyalkyl, C₁₋₅ alkoxyalkyl, C₁₋₅ carboxyalkyl, acetoxyalkyl, aryloxyalkyl, R(CH₂CH₂O)ₙ, where n=1-5, and R═C₁₋₂ alkyl, phenyl, hydroxyl, carboxyl;
R⁶ and R⁹ are independently selected from acyl, acyloxy, optionally substituted alkoxy, optionally substituted alkyl, optionally substituted amino, optionally substituted aryl, optionally substituted aryloxy, carboxyl, (optionally substituted alkoxy)carbonyl, (optionally substituted amino)carbonyl, (optionally substituted amino)carbonyloxy, cyano, fluoro, chloro, hydrogen, hydroxyl, and nitro;
R⁵, R¹⁰, R¹¹ and R¹² are independently selected from H, OH, C₁₋₂ alkyl, C₁₋₃ acyl, and phenyl;
R²¹, R²², R²³ and R²⁴ are optional substituents, and when present are independently selected from H, OH, optionally substituted C₁₋₄ alkyl, optionally substituted C₃₋₁₀ alkyl, optionally substituted aryl, optionally substituted heteroaryl, optionally substituted heterocyclyl;
alternatively, at least one of
   R¹ and R²¹,
   R¹¹ and R²,
   R³ and R²²,
   R¹² and R^{1a},
   R^{4a} and R⁵,
   R²³ and R⁶,
   R⁹ and R²⁴, and
   R¹⁰ and R⁴ respectively taken together represent O(CHR³²)₁₋₄ or (CHR³²)₁₋₄, wherein R³² represents H, methyl, or ethyl;
with the proviso that at least one of
   R¹ and R²¹,
   R¹¹ and R²,
   R³ and R²²,
   R¹² and R^{1a},
   R^{4a} and R⁵,
   R²³ and R⁶,
   R⁹ and R²⁴, and
   R¹⁰ and R⁴ respectively taken together represent O(CHR³²)₂₋₄ or (CHR³²)₂₋₄, wherein R³² represents H, methyl, or ethyl.

A further preferred embodiment of the present invention provides a compound of Formula I of claim 9. A compound of Formula I of claim 8: 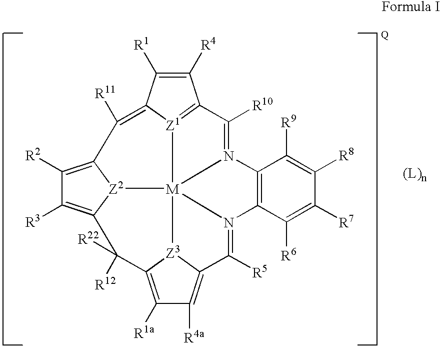
its hydrate, pharmaceutically acceptable salt or prodrug form thereof, wherein:
M represents Lu(III) or Gd(III);
Z¹, Z² and Z³ independently represent N;
L represents OAc;
R¹ represents (CH₂)₃OH;
R² and R³ represent CH₃;
R⁴ and R^{4a} represent CH₃;
R⁵ and R⁶ represent H;
R⁷, and R⁸ are independently selected from O—(CH₂)₃—OH, OCH₃ and H;
R⁹ represents H; and
R¹² and R^{1a} taken together represent O(CHR³²)₃ wherein R³² represents H.

The present invention, in one of its aspects, provides a pharmaceutical composition, comprising a pharmaceutically acceptable carrier and a therapeutically effective amount of a compound of Formula I, its hydrate, its prodrug or a pharmaceutically acceptable salt form thereof.

Yet another aspect of the present invention provides a method of treating a host harboring a neoplasm or atheroma, comprising administering to the host a compound of Formula I, and administering ionizing radiation to the host in proximity to the neoplasm or atheroma.

Experimental

General

All reagents were used as supplied commercially unless otherwise noted. Motexafin gadolinium (a.k.a. gadolinium Texaphyrin, Compound 1) was prepared according to the literature procedure of Sessler et al. (Inorg. Chem., Vol. 32, pg. 3175 (1993); J. Phys. Chem., Vol. 103, pgs. 787-794 (1999)). UV-VIS spectra were measured on a Hewlett-Packard 8452A Diode Array spectrophotometer. ¹H and ¹³C NMR spectra, COSY, and HMQC were performed using a Varian XL-400 instrument using a deuterated solvent as specified. Some ¹H and ¹³C NMR spectra were also obtained using a GE 300 instrument. FAB and FAB high resolution mass spectra were performed by University of California at Berkeley, Mass Spectrometry Facility.
Preparation of Gadolinium Oxotexaphlorin (2): 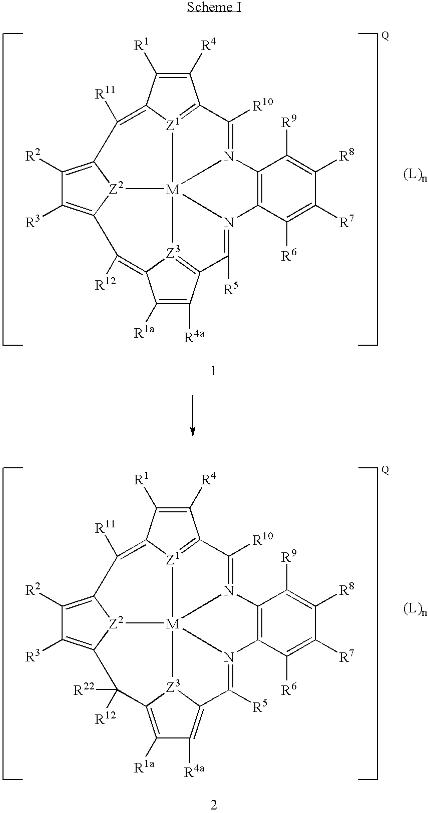
In compound 2, R¹² and R²² along with the carbon atom to which they are attached represent C═O.

In a 250 mL round-bottom flask, equipped with a magnetic stirrer bar, was placed a mixture of Compound 1 (1, 230.0 mg, 0.20 mmol) and anhydrous CH₂Cl₂ (100 mL). The mixture was agitated at ambient temperature. To the agitating mixture then was added MeONa (43.2 mg, 0.80 mmol) in one portion. The resulting reaction mixture was agitated at ambient temperature for about 12 hours, and then concentrated under reduced pressure to yield a residue. The residue was purified using column chromatography on silica gel column (eluent: MeOH/CH₂Cl₂=7/100 (v/v)) to yield a compound of Formula I (compound 2) as a deep green solid (73 mg, 33%).

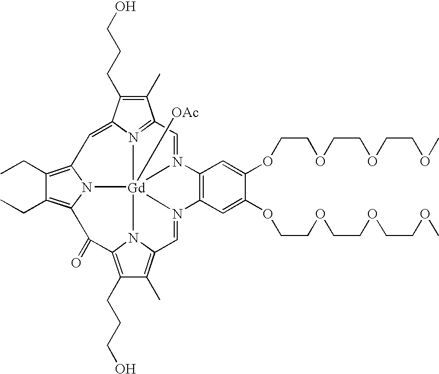

This compound was prepared using the procedure out lined in Scheme I above.

FAB MS (m/z): 1045 [M-OAc⁻], 985, 913, 897, 659, 443.

HRMS: calcd. for C₄₈H₆₅¹⁵⁸ GdN₅O₁₁ [M-OAc⁻]: 1045.3933; found: 1045.3962.

UV-VIS (CH₃OH): 330, 432, 630, 672 nm.

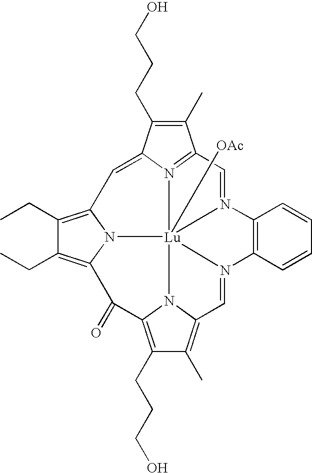

This compound was prepared using the procedure outlined in Scheme I above, and which is discussed below.

In a 250 mL round-bottom flask, equipped with a magnetic stirrer bar, was placed a mixture of Motexafin lutetium (compound 3, 168 mg, 0.20 mmol) and anhydrous CH₂Cl₂ (100 mL). The mixture was agitated at ambient temperature. To the agitating mixture then was added MeONa (43.2 mg, 0.80 mmol) in one portion. The resulting reaction mixture was agitated at ambient temperature for about 12 hours, and then concentrated under reduced pressure to yield a residue. The residue was purified using column chromatography on silica gel column (eluent: MeOH/CH₂Cl₂=5/100 (v/v)) to yield compound 4 as a deep green solid (54 mg, 34%).

¹H NMR (400 MHz, CD₃OD): 9.91(s, 1H), 9.85(s, 1H), 8.45(d, J=6.6 Hz, 1H), 8.41(d, J=6.6 Hz, 1H), 8.18(s, 1H), 7.59(t, J=6.6 Hz, 1H), 7.53(t, J=6.6 Hz, 1H), 3.62-3.55(m, 4H), 3.11(t, J=5.7 Hz, 2H), 3.08-2.95(m, 6H), 2.55(s, 3H), 2.53(s, 3H), 1.92-1.84(m, 4H), 1.52(brs, 3H), 1.29(t, J=6.0 Hz, 3H), 1.25(t, J=5.9 Hz, 3H) ppm.

¹³C NMR (400 MHz, CD₃OD): 183.60, 182.81, 161.40, 152.99, 150.49, 149.03, 144.85, 144.33, 143.79, 142.99, 142.56, 142.08, 141.27, 136.44, 133.06, 131.89, 129.49, 127.78, 127.23, 118.48, 118.11, 62.82, 62.18, 35.87, 34.17, 23.57, 23.41, 22.06, 21.60, 19.46, 18.37, 16.10, 9.63, 9.89 ppm.

FAB MS (m/z): 738 [M-OAc⁻], 676, 648, 573, 487.

HRMS: calcd. for C₃₄H₃₇LuN₅O₃ [M-OAc⁻]: 738.2303; found: 738.2301.

UV-VIS (CH₃OH): 326, 424, 654 nm.
Preparation of Compounds of Formula I:

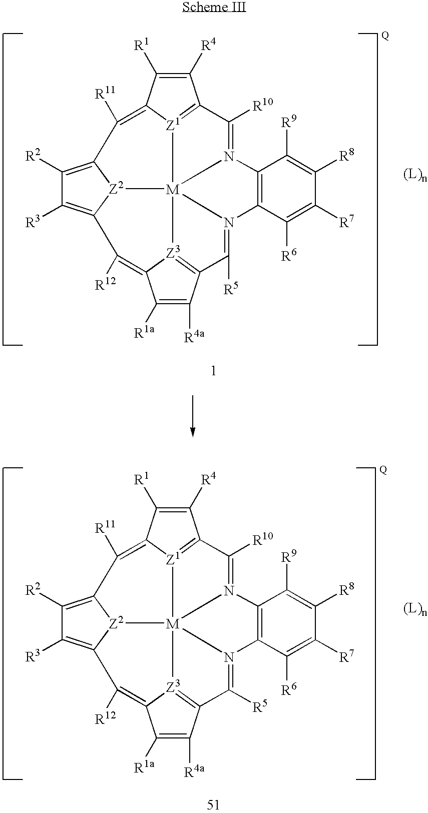

In compound 51 (compound of Formula I) R¹² and R^{1a} taken together represent W(CHR³²)₁₋₈, where W represents O and R³² represent H.

A mixture of compound 1, (Compound 1, 3 g, 2.6 mmol) and CH₃OH (150 mL) was agitated at ambient temperature in a 250 mL round bottom flask. To the agitating mixture then was added CH₃ONa (10% methanol solution, 5 mL) in one portion. The resulting reaction mixture then was agitated for about 3 hours at ambient temperature. The agitated reaction mixture then was neutralized with HOAC solution to a pH of from about 6 to about 7. The neutralized reaction mixture then was concentrated under reduced pressure to afford a residue. The residue was subjected to column chromatography on silica gel column (eluent:

MeOH/CH₂Cl₂=5/100 (v/v)) to yield compound 51.

MS (m/z): 1087.4 [M-OAc⁻]⁺, 983.3, 955.1, 833.2.

UV-vis (CH₃OH): 460, 716 nm.

### Example 52

### This compound was prepared using the procedure of Scheme III and Example 51 above.

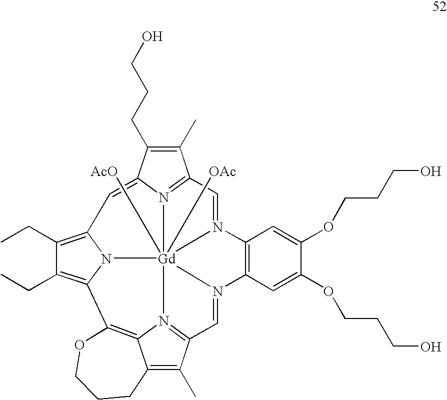 Preparation of Lu-Compounds of Formula I

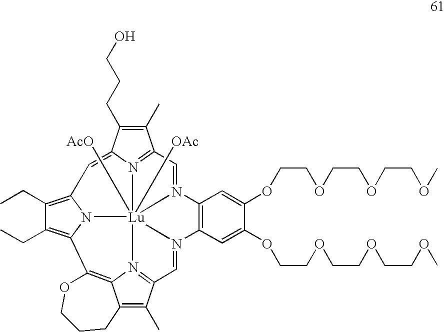

This compound was prepared using the procedure of Scheme III that is outlined below.

A mixture of a compound of Motexafin Lutecium (Compound 3, 3 g, 2.57 mmol) and CH₃OH (150 mL) was agitated at ambient temperature in a 250 mL round bottom flask. To the agitating mixture then was added CH₃ONa (10% methanol solution, 5 mL) in one portion. The resulting reaction mixture then was agitated for about 12 hours at ambient temperature. The agitated reaction mixture then was neutralized with HOAc solution to a pH of from about 6 to about 7. The neutralized reaction mixture then was concentrated under reduced pressure to afford a residue. The residue was subjected to column chromatography on silica gel column (eluent: MeOH/CH₂Cl₂=5/100 (v/v)) to yield compound 61.

MS (m/z): 1104.4 [M-OAc⁻]⁺, 1076.3, 1000.3, 972.4.

UV-vis (CH₃OH): 462, 706 nm.
The following compounds were prepared using the procedure outlined in Scheme IV above:

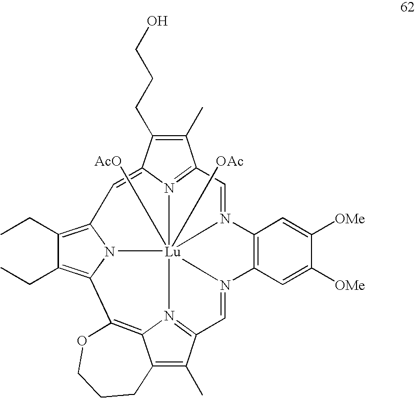

¹H NMR(300 MHz/CDCl₃—CD₃OD) 11.12(s, 1H), 11.05(s, 1H), 9.64(s, 1H), 8.60(s, 1H), 8.55(s, 1H), 5.13(t, J=6.0 Hz, 2H), 3.75(t, J=5.8 Hz, 2H), 3.70-3.52(m, 8H), 3.40(s, 3H), 3.46(s, 3H), 2.95-2.70(m, 8H), 2.24(quint, J=6.4 Hz, 2H), 1.67(t, J=7.2 Hz, 3H), 1.63(t, J=7.2 Hz, 3H) ppm.

ESI MS (m/z): 840.3 [M-OAc]⁺.

FAB MS (m/z): 781 [M-2OAc]⁺.

HRMS: calcd. for C₃₆H₄₀LuN₅O₄ [M-2OAc]⁺: 781.2488; found: 781.2469.

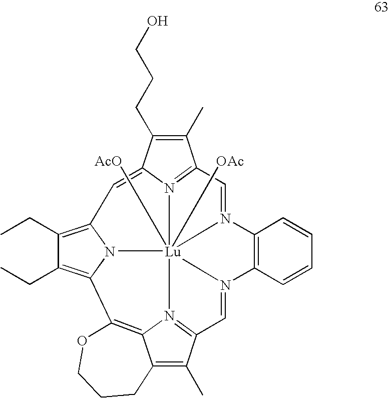

¹H NMR(300 MHz/CD₂Cl₂—CD₃OD) 10.99(s, 1H), 10.97(s, 1H), 9.78-9.61(m, 3H), 8.25-8.19(m, 2H), 5.04(t, J=5.8 Hz, 2H), 3.67(t, J=6.4 Hz, 2H), 3.54-3.39(m, 8H), 2.96(s, 3H), 2.80(s, 3H), 2.78-2.69(m, 2H), 2.16(quint, J=6.7 Hz, 2H), 1.57(t, J=7.9 Hz, 3H), 1.54(t, J=7.6 Hz, 3H) ppm.

¹³C NMR(300 MHz/CD₂Cl₂—CD₃OD) 156.39, 148.26, 143.55, 138.96, 137.81, 137.71, 137.24, 136.40, 135.09, 134.59, 134.49, 133.47, 132.39, 132.04, 124.68, 124.08, 121.60, 120.16, 119.71, 119.42, 114.58, 108.89, 68.76, 51.68, 24.95, 20.06, 15.49, 12.95, 12.22, 11.39, 9.63, 8.39, 6.20, 0.86 ppm.

FAB MS (m/z): 721 [M-2OAc]⁺.

HRMS: calcd. for C₃₄H₃₆LuN₅O₂ [M-2OAc]⁺: 721.2276; found: 721.2252.

Preparation of the Starting Materials, Compound 1 and Compound 3

Compounds 1 and 3 were prepared by the procedure of Sessler et al. (Inorg. Chem., Vol. 32, pg. 3175 (1993); J. Phys. Chem., Vol. 103, pgs. 787-794 (1999)).

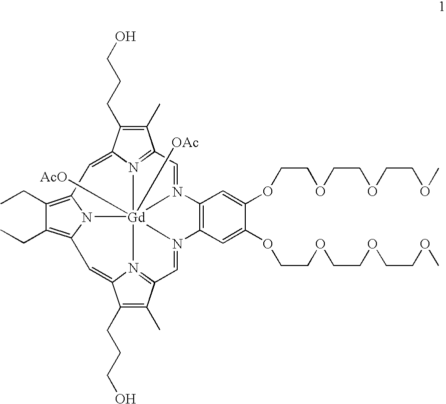

Elemental Analysis:

Calculated: C, 54.34; H, 6.27; N, 6.10, Gd 13.69

Found: C, 54.32; H, 6.40; N, 6.11, Gd 13.71

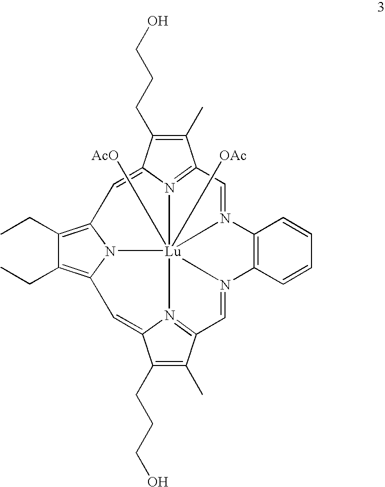

¹H NMR (400 MHz, CD₃OD): 11.55(s, 2H), 9.61(dd, J=5.1, 2.6 Hz, 2H), 9.59(s, 2H), 8.32(dd, J=5.1, 2.6 Hz, 2H), 3.62(t, J=5.2 Hz, 4H), 3.57(t, J=5.2 Hz, 4H), 3.50(q, J=6.0 Hz, 4H), 3.05(s, 6H), 2.13(quint, J=5.2 Hz, 4H), 1.55(t, J=6.0 Hz, 6H), 0.55(brs, 6H) ppm.

¹³C NMR (400 MHz, CD₃OD) 179.33, 158.00, 151.44, 147.75, 146.88, 145.57, 144.22, 141.81, 135.64, 129.73, 119.76, 118.84, 60.74, 34.42, 22.10, 21.79, 19.25, 17.86, 10.47 ppm.

ESI MS (m/z): 782 [M-OAc^{-].}

HRMS: calcd. for C₃₄H₃₈LuN₅O₂⁺[M-2OAc]⁺: 723.2433; found: 723.2460.

UV-VIS (CH₃OH): 333, 436, 762 nm.

Utility

The compounds of the present invention are effective in the treatment of conditions known to respond to metallotexaphyrin therapy, including diseases characterized by neoplastic tissue, (e.g. the cancers sarcoma, lymphoma, leukemia, carcinoma, brain metastases, glioma, glioblastoma, cancer of the prostate, melanoma, and the like), cardiovascular diseases (e.g., atherosclerosis, intimal hyperplasia and restenosis) and other activated macrophage-related disorders including autoimmune diseases (e.g., rheumatoid arthritis, Sjogrens, scleroderma, systemic lupus erythematosus, non-specific vasculitis, Kawasaki's disease, psoriasis, Type I diabetes, pemphigus vulgaris, multiple sclerosis), granulomatous diseases (e.g., tuberculosis, sarcoidosis, lymphomatoid granulomatosis, Wegener's granulomatosus), inflammatory diseases (e.g., inflammatory lung diseases such as interstitial pneumonitis and asthma, inflammatory bowel diseases such as Crohn's disease, and inflammatory arthritis), in transplant rejection (e.g., in heart/lung transplants) and in ophthalmic diseases that result from undesired neovascularization, in particular age-related macular degeneration.

Definitions

As used in the present specification, the following words and phrases are generally intended to have the meanings as set forth below, except to the extent that the context in which they are used indicates otherwise.

The term “optional” or “optionally” means that the subsequently described event or circumstance may or may not occur, and that the description includes instances where said event or circumstance occurs and instances in which it does not. For example, “optionally substituted alkyl” means either “alkyl” or “substituted alkyl,” as defined below. It will be understood by those skilled in the art with respect to any group containing one or more substituents that such groups are not intended to introduce any substitution or substitution patterns (e.g., substituted alkyl includes optionally substituted cycloalkyl groups, which in turn are defined as including optionally substituted alkyl groups, potentially ad infinitum) that are sterically impractical and/or synthetically non-feasible. Thus, the substituents described for R¹ to R¹² should be generally understood as having a maximum molecular weight of about 1,000 daltons, and more typically, up to about 500 daltons (except in those instances where macromolecular substituents are clearly intended, e.g., polypeptides, polyethylene glycols, DNA, RNA and the like).

The term “acyl” refers to the groups —C(O)—H, —C(O)-(optionally substituted alkyl), —C(O)-(optionally substituted cycloalkyl), —C(O)-(optionally substituted alkenyl), —C(O)-(optionally substituted cycloalkenyl), —C(O)-(optionally substituted aryl), —C(O)-(optionally substituted heteroaryl) and —C(O)-(optionally substituted heterocyclyl).

The term “acyloxy” refers to the moiety —O-acyl, including, for example, —O—C(O)-alkyl.

The term “alkoxy” refers to the groups —O-alkyl, —O-alkenyl, —O-cycloalkyl, —O-cycloalkenyl, and —O-alkynyl. Preferred alkoxy groups are —O-alkyl and include, by way of example, methoxy, ethoxy, n-propoxy, iso-propoxy, n-butoxy, tert-butoxy, sec-butoxy, n-pentoxy, n-hexoxy, 1,2-dimethylbutoxy, and the like.

The term “substituted alkoxy” refers to the groups —O-(substituted alkyl), —O-(substituted a alkenyl), —O-(substituted cycloalkyl), —O-(substituted cycloalkenyl), —O-(substituted alkynyl) and —O-(optionally substituted alkylene)-alkoxy. One preferred substituted alkoxy group is “polyalkoxy” or —O-(substituted alkylene)-alkoxy, and includes groups such as —OCH₂CH₂OCH₃, and (or PEG) groups such as —O(CH₂CH₂O)ₓCH₃, where x is an integer of about 2-20, preferably about 2-10, and more preferably about 2-5. Another preferred substituted alkoxy group is —O-(substituted alkyl), and includes groups such as —OCH₂(CH₂)_{y}OH, where y is an integer of about 1-10, preferably about 1-4.

The term “alkoxyalkylene” refers to the groups: -alkylene-O-alkyl, -alkylene-O-(substituted alkyl), -(substituted alkylene)-O-alkyl and -(substituted alkylene)-O-(substituted alkyl). A preferred alkoxyalkylene group is -alkylene-O-alkyl and include, by way of example, methoxymethylene (—CH₂OCH₃), methoxyethylene (—CH₂CH₂OCH₃), n-(iso-propoxy)propylene [—CH₂CH₂CH₂OCH(CH₃)₂] and the like.

The term “alkenyl” refers to a monoradical of a branched or unbranched unsaturated hydrocarbon group preferably having from 2 to 20 carbon atoms, more preferably 2 to 10 carbon atoms and even more preferably 2 to 6 carbon atoms and having at least 1 and preferably from 1-6 sites of vinyl unsaturation. Preferred alkenyl groups include ethenyl (—CH═CH₂), 2-propen-1-yl (—CH₂CH═CH₂), isopropenyl [—C(CH₃)═CH₂], and the like.

The term “substituted alkenyl” refers to an alkenyl group in which 1 or more (up to about 5, preferably up to about 3) hydrogen atoms is replaced by a substituent independently selected from the group: ═O, ═S, acyl, acyloxy, optionally substituted alkoxy, optionally substituted amino, optionally substituted aryl, optionally substituted aryloxy, carboxyl, (optionally substituted alkoxy)carbonyl, (optionally substituted amino)carbonyl, (optionally substituted alkoxy)carbonyloxy, (optionally substituted amino)carbonyloxy, cyano, optionally substituted cycloalkyl, halogen, optionally substituted heteroaryl, optionally substituted heteroaryloxy, optionally substituted heterocyclyl, optionally substituted heterocyclooxy, hydroxyl, nitro, optionally substituted phosphine, optionally substituted azo, phosphonato, phosphono, sulfanyl, sulfinyl, and sulfonyl.

The term “alkenylene” refers to a diradical derived from the above-defined monoradical, alkenyl. This term is exemplified by groups such as ethenylene (—CH═CH—), the propenylene isomers (e.g., —CH₂CH═CH— and —C(CH₃)═CH—) and the like.

The term “substituted alkenylene” refers to a diradical derived from the above-defined monoradical, substituted alkenyl.

The term “alkyl” refers to a monoradical branched or unbranched saturated hydrocarbon chain preferably having from about 1 to 20 carbon atoms, more preferably about 1 to 10 carbon atoms, and even more preferably about 1 to 6 carbon atoms. This term is exemplified by groups such as methyl, ethyl, n-propyl, iso-propyl, n-butyl, iso-butyl, n-hexyl, n-decyl, tetradecyl, and the like.

The term “substituted alkyl” refers to an alkyl group in which 1 or more (up to about 5, preferably up to about 3) hydrogen atoms is replaced by a substituent independently selected from the group: ═O, ═S, acyl, acyloxy, optionally substituted alkoxy, optionally substituted amino, optionally substituted aryl, optionally substituted aryloxy, carboxyl, (optionally substituted alkoxy)carbonyl, (optionally substituted amino)carbonyl, (optionally substituted alkoxy)carbonyloxy, (optionally substituted amino)carbonyloxy, cyano, optionally substituted cycloalkyl, halogen, optionally substituted heteroaryl, optionally substituted heteroaryloxy, optionally substituted heterocyclyl, optionally substituted heterocyclooxy, hydroxyl, nitro, optionally substituted phosphine, phosphonato, phosphono, sulfanyl, sulfinyl, and sulfonyl.

One of the preferred optional substituents for alkyl is hydroxy, exemplified by hydroxyalkyl groups, such as 2-hydroxyethyl, 3-hydroxypropyl, 3-hydroxybutyl, 4-hydroxybutyl, and the like; dihydroxyalkyl groups (glycols), such as 2,3-dihydroxypropyl, 3,4-dihydroxybutyl, 2,4-dihydroxybutyl, and the like; and those compounds known as polyethylene glycols, polypropylene glycols and polybutylene glycols, and the like.

The term “alkylene” refers to a diradical derived from the above-defined monoradical, alkyl. This term is exemplified by groups such as methylene (—CH₂—), ethylene (—CH₂CH₂—), the propylene isomers [e.g., —CH₂CH₂CH₂— and —CH(CH₃)CH₂—] and the like.

The term “substituted alkylene” refers to a diradical derived from the above-defined monoradical, substituted alkyl. Examples of substituted alkylenes are chloromethylene (—CH(Cl)—), aminoethylene (—CH(NH₂)CH₂—), methylaminoethylene (—CH(NHMe)CH₂—), 2-carboxypropylene isomers (—CH₂CH(CO₂H)CH₂—), ethoxyethylene (CH(OCH₂CH₃)CH₂), 3-oxapentylene (—CH₂CH₂O—CH₂CH₂—), N-methyl-3-azapentylene (—CH₂CH₂N(CH₃)CH₂CH₂—), 3,6,9-trioxaundecylene (2-ethoxy-ethoxy)ethylene (—CH₂CH₂O—CH₂CH₂—OCH₂CH₂—OCH₂CH₂—), and the like.

The term “alkynyl” refers to a monoradical of an unsaturated hydrocarbon, preferably having from 2 to 20 carbon atoms, more preferably 2 to 10 carbon atoms and even more preferably 2 to 6 carbon atoms and having at least 1 and preferably from 1-6 sites of acetylene (triple bond) unsaturation. Preferred alkynyl groups include ethynyl, (—C≡CH), propargyl (or propynyl, —C═CCH₃), and the like.

The term “substituted alkynyl” refers to an alkynyl group in which 1 or more (up to about 5, preferably up to about 3) hydrogen atoms is replaced by a substituent independently selected from the group: ═O, ═S, acyl, acyloxy, optionally substituted alkoxy, optionally substituted amino, optionally substituted aryl, optionally substituted aryloxy, carboxyl, (optionally substituted alkoxy)carbonyl, (optionally substituted amino)carbonyl, (optionally substituted alkoxy)carbonyloxy, (optionally substituted amino)carbonyloxy, cyano, optionally substituted cycloalkyl, halogen, optionally substituted heteroaryl, optionally substituted heteroaryloxy, optionally substituted heterocyclyl, optionally substituted heterocyclooxy, hydroxyl, nitro, optionally substituted phosphine, optionally substituted azo, phosphonato, phosphono, sulfanyl, sulfinyl, and sulfony.

The term “alkynylene” refers to a diradical derived from the above-defined monoradical, alkynyl. Preferred alkynylene groups include ethynylene (—C—C—), propargylene (—CH₂—C═C—) and the like.

The term “substituted alkynylene” refers to a diradical derived from the above-defined monoradical, substituted alkynyl.

The term “amino” refers to the group —NH₂.

The term “substituted amino” refers to the group —NHR or —NRR where each R is independently selected from the group: acyl, optionally substituted alkenyl, optionally substituted alkyl, optionally substituted alkoxy, optionally substituted alkoxycarbonyl, optionally substituted alkynyl, optionally substituted aminocarbonyl, optionally substituted aryl, carboxy, optionally substituted cycloalkyl, optionally substituted heteroaryl, and optionally substituted heterocyclyl. Preferred amino substituents include optionally substituted alkyl, aryl, optionally substituted alkoxycarbonyl (also referred to as a “carbamate”), optionally substituted aminocarbonyl (also referred to as a urea) and heteroaryl.

The term “apical ligand” refers to an anion that binds to the core metal of the metallotexaphyrin, e.g., with de-localized electrostatic or weak coordinate-covalent bonds. The number of apical ligands (n) is defined as an integer of 0-5. It should be noted that the apical ligands act to neutralize the charge on the metallotexaphyrin. Thus, typically n is 1 when M is a divalent cation, and n is 2 when M is a trivalent cation (because the core itself neutralizes one unit charge). However, if any of R¹, R¹′, R², R³, R⁴, R⁴′, R⁵, R⁶, R⁷, R⁸, R⁹, R¹⁰, R¹¹ and R¹² is capable of forming an acid addition salt, for example a carboxylate or a phosphate, then n will decrease appropriately. It is also possible that the apical ligands could have two functionalities capable of forming an anion, for example a dicarboxylic acid, and such ligands are intended to be within the scope of the invention. In general, any molecule containing a carboxylic acid or phosphate may be used as an apical ligand, for example biomolecules, including lipoproteins, estradiol and amino acids, carboxylates of sugar derivatives, such as gluconic acid or glucoronic acid, cholesterol derivatives such as cholic acid and deoxycholic acid, PEG acids, organophosphates, such as methylphosphonic acid and phenylphosphonic acid, and phosphoric acid or other inorganic acids, and the like, or sulfonic acid derivatives such as methanesulfonic acid, ethanesulfonic acid or “carboxylic acid derivatives”, which term refers to compounds of the formula R-CO₂H, in which R is optionally substituted alkyl, optionally substituted alkenyl, optionally substituted alkynyl, or optionally substituted aryl, as defined above. Preferred are gluconic and glucuronic acid, and those carboxylic acid derivatives where R is optionally substituted alkyl, for example acids of 1-20 carbon atoms, such as formic acid, acetic acid, propionic acid, butyric acid, pentanoic acid, 3,6,9-trioxodecanoic acid, 3,6-dioxoheptanoic acid, methylvaleric acid, glycolic acid, pyruvic acid, oxalic acid, malic acid, malonic acid, succinic acid, maleic acid, fumaric acid, tartaric acid, citric acid, and the like. Also preferred are those carboxylic acid derivatives where R is aryl, in particular where R is optionally substituted phenyl, for example benzoic acid, salicylic acid, 3-fluorobenzoic acid, 4-aminobenzoic acid, cinnamic acid, mandelic acid, p-toluene-sulfonic acid, and the like.

The term “aromatic” refers to a cyclic or polycyclic moiety having a conjugated unsaturated (4n+2)π electron system (where n is a positive integer), sometimes referred to as a delocalized π electron system.

The term “aryl” refers to an aromatic cyclic hydrocarbon group of from 0.6 to 20 carbon atoms having a single ring (e.g., phenyl) or multiple condensed (fused) rings (e.g., naphthyl or anthryl). Preferred aryls include phenyl, naphthyl and the like.

The term “substituted aryl” refers to an aryl group in which 1 or more (up to about 5, preferably up to about 3) hydrogen atoms is replaced by a substituent independently selected from the group: ═O, ═S, acyl, acyloxy, optionally substituted alkoxy, optionally substituted amino, optionally substituted a ryl, optionally substituted aryloxy, carboxyl, (optionally substituted alkoxy)carbonyl, (optionally substituted amino)carbonyl, (optionally substituted alkoxy)carbonyloxy, (optionally substituted amino)carbonyloxy, cyano, optionally substituted cycloalkyl, halogen, optionally substituted heteroaryl, optionally substituted heteroaryloxy, optionally substituted heterocyclyl, optionally substituted heterocyclooxy, hydroxyl, nitro, optionally substituted phosphine, optionally substituted azo, phosphonato, phosphono, sulfanyl, sulfinyl, and sulfony (except as otherwise constrained by the definition for the aryl substituent).

The term “aryloxy” refers to the group —O-aryl.

The term “substituted aryloxy” refers to the group —O-(substituted aryl).

The term “arylalkyl” refers to the moiety “-alkylene-aryl” each having the meaning as defined herein. Such arylalkyl groups are exemplified by benzyl, phenethyl, 3-naphthylpropyl and the like. Arylalkyl moieties also fall within the definition of optionally substituted alkyl, e.g., as a 2-phenyl-n-pentyl moiety.

The term “substituted arylalkyl” refers to the moiety “-(optionally substituted alkylene)-(optionally substituted aryl)”, each having the meaning as defined herein, where at least one of the aryl or alkylene groups is substituted, e.g., 4-(N-methyl-pyrrolyl)pentylene.

The term “carbonyl” refers to the di-radical “—C(═O)-”, which is also written as “—C(O)-”.

The term “(optionally substituted alkoxy)carbonyl” refers to the groups: —C(O)O-(optionally substituted alkyl), —C(O)O-(optionally substituted cycloalkyl), —C(O)O-(optionally substituted alkenyl), and —C(O)O-(optionally substituted alkynyl). These moieties are also referred to as esters.

The term “(optionally substituted amino)carbonyl” refers to the group —C(O)-(optionally substituted amino). This moiety is also referred to as a primary, secondary or tertiary carboxamide.

The term “(optionally substituted alkyl)carbonyloxy” refers to the group —O—C(O)-(optionally substituted alkyl). This moiety is also referred to as a “carbonate.”

The term “(optionally substituted amino)carbonyloxy” refers to the group —O—C(O)-(optionally substituted amino). This moiety is also referred to as a “carbamate.”

The term “carboxy” or “carboxyl” refers to the moiety “—C(O)OH”, which is also illustrated as “—COOH”.

The term “compound of Formula I” is intended to encompass the metallotexaphyrins of the invention as disclosed, coordination complexes of the compounds of Formula I, and/or the pharmaceutically acceptable salts of such compounds. In addition, the compounds of this invention include the individual stereochemical isomers and mixtures thereof, arising from the selection of substituent groups.

The term “cycloalkyl” refers to non-aromatic cyclic hydrocarbon groups having about 3 to 40 (preferably about 4 to 15) carbon atoms having a single ring or multiple condensed rings. Such cycloalkyl groups include, by way of example, single ring structures such as cyclopropyl, cyclobutyl, cyclopentyl, cyclooctyl, and the like, or multiple ring structures such as adamantanyl, and the like.

The term “substituted cycloalkyl” refers to a cycloalkyl group in which 1 or more (up to about 5, preferably up to about 3) hydrogen atoms is replaced by a substituent independently selected from the group: ═O, ═S, acyl, acyloxy, optionally substituted alkoxy, optionally substituted amino, optionally substituted aryl, optionally substituted aryloxy, carboxyl, (optionally substituted alkoxy)carbonyl, (optionally substituted amino)carbonyl, (optionally substituted alkoxy)carbonyloxy, (optionally substituted amino)carbonyloxy, cyano, optionally substituted cycloalkyl, halogen, optionally substituted heteroaryl, optionally substituted heteroaryloxy, optionally substituted heterocyclyl, optionally substituted heterocyclooxy, hydroxyl, nitro, optionally substituted phosphine, phosphonato, optionally substituted azo, phosphono, sulfanyl, sulfinyl, and sulfony (except as otherwise constrained by the definition for the cycloalkyl substituent).

The term “cycloalkylene” refers to a diradical derived from the above-defined monoradical, cycloalkyl, and is exemplified by 1,1-cyclopropylene, 1,2-cyclobutylene, 1,4-cyclohexylene and the like.

The term “substituted cycloalkylene” refers to the diradical derived from substituted cycloalkyl as defined above.

The term “halo” or “halogen” refers to fluoro, chloro, bromo and iodo.

The term “heteroaryl” refers to an aromatic cyclic hydrocarbon group having about 1 to 40 (preferably from about 3 to 15) carbon atoms and about 1 to 10 hetero atoms (preferably about 1 to 4 heteroatoms, selected from nitrogen, sulfur, phosphorus, and/or oxygen) within at least one ring. Such heteroaryl groups can have a single ring (e.g., pyridyl or furyl) or multiple condensed rings (e.g., indolizinyl or benzothienyl). Preferred heteroaryls include pyridyl, pyrrolyl and furyl.

The term “substituted heteroaryl” refers to a heteroaryl group in which 1 or more (up to about 5, preferably up to about 3) hydrogen atoms is replaced by a substituent independently selected from the group: ═O, ═S, acyl, acyloxy, optionally substituted alkoxy, optionally substituted amino, optionally substituted aryl, optionally substituted aryloxy, carboxyl, (optionally substituted alkoxy)carbonyl, (optionally substituted amino)carbonyl, (optionally substituted alkoxy)carbonyloxy, (optionally substituted amino)carbonyloxy, cyano, optionally substituted cycloalkyl, halogen, optionally substituted heteroaryl, optionally substituted heteroaryloxy, optionally substituted heterocyclyl, optionally substituted heterocyclooxy, hydroxyl, nitro, optionally substituted phosphine, optionally substituted azo, phosphonato, phosphono, sulfanyl, sulfinyl, and sulfony (except as otherwise constrained by the definition for the heteroaryl substituent).

The term “heteroaryloxy” refers to the group —O-heteroaryl.

The term “heteroarylene” refers to the diradical group derived from heteroaryl (including substituted heteroaryl), as defined above, and is exemplified by the groups 2,6-pyridylene, 2,4-pyridylene, 1,2-quinolinylene, 1,8-quinolinylene, 1,4-benzofuranylene, 2,5-pyridylene, 2,5-indolylene and the like.

The terms “heterocycle”, “heterocyclic” and “heterocyclyl” are interchangeable, and refer to a monoradical, saturated or unsaturated, non-aromatic cyclic hydrocarbon group having from about 3 to about 40 (preferably from about 3 to about 15) carbon atoms wherein one to about 10 carbon atoms are independently replaced hetero atoms selected from nitrogen, sulfur, phosphorus, oxygen, and selenium. In a preferred embodiment about 1 to about 4 carbon atoms are replaced by hetero atoms. Such heterocyclic groups can have a single ring or multiple condensed rings. Illustrative examples of a heterocycle are morpholino, piperidinyl, and the like.

The terms “substituted heterocycle”, “substituted heterocyclic” and “substituted heterocyclyl” refer to a heterocyclyl group in which 1 or more (up to about 5, preferably up to about 3) hydrogen atoms is replaced by a substituent independently selected from the group: ═O, ═S, acyl, acyloxy, optionally substituted alkoxy, optionally substituted amino, optionally substituted aryl, optionally substituted aryloxy, carboxyl, (optionally substituted alkoxy)carbonyl, (optionally substituted amino)carbonyl, (optionally substituted alkoxy)carbonyloxy, (optionally substituted amino)carbonyloxy, cyano, optionally substituted cycloalkyl, halogen, optionally substituted heteroaryl, optionally substituted heteroaryloxy, optionally substituted heterocyclyl, optionally substituted heterocyclooxy, hydroxyl, nitro, optionally substituted phosphine, phosphonato, optionally substituted azo, phosphono, sulfanyl, sulfinyl, and sulfony (except as otherwise constrained by the definition for the heterocyclic substituent).

The term “heterocyclylooxy” refers to the group —O-heterocycle.

The term “heterocyclylene” refers to the diradical group formed from a heterocycle, as defined herein, and is exemplified by the groups 2,6-morpholino, 2,5-morpholino and the like.

The term “linker” as used herein means a covalent connection of a functional group (e.g., a site directing group, a catalytic group or a chemotherapeutic agent) to a metallotexaphyrin or its analogue, and may be, for example, a covalent bond or an alkylene, alkenylene, alkynylene, arylene, ether, PEG moiety, and the like, all of which may be optionally substituted. Examples of reactions to form a covalent link include the reaction between an amine (on either the functional group or the linker precursor) with a carboxylic acid (on the other) to form an amide link. Similar reactions well known in the art are described in standard organic chemistry texts such as J. March, “Advanced Organic Chemistry”, 4^{th} Edition, (Wiley-Interscience (New York)), 1992.

Dashed lines in cyclic structures indicate optional unsaturation without violating valency rules. Thus in the following structure 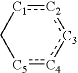
the dashed lines between C₁ and C₂, C₂ and C₃, and C₄ and C₅ respectively indicate that a double bond may or may not exist between all or just a couple of carbon atoms numbered C₁ and C₂, C₂ and C₃, and C₄ and C₅ respectively, as long as the valency rules are not violated.

The term “macrocycle” as used herein refers to a class of polypyrrolic macrocycles that are capable of forming stable complexes with metals by incorporating a metal (as its cation) within a central binding cavity (core) of the macrocycle, and the anions associated with the metal cation are found above and below the core; these anions are known as apical ligands. This class of macrocycles includes porphyrins, the so-called “expanded porphyrins”, and similar structures. Specific examples are porphyrins, porphyrin isomers, porphyrin-like macrocycles, benzophyrins, texaphyrins, alaskaphyrins, sapphyrins, rubyrins, porphycenes, chlorins, benzochlorins, and purpurins.

As used herein, “pharmaceutically acceptable carrier” or “pharmaceutically acceptable excipient” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. Supplementary active ingredients can also be incorporated into the compositions.

The term “pharmaceutically acceptable salt” refers to salts which retain the biological effectiveness and properties of the compounds of this invention and which are not biologically or otherwise undesirable. In many cases, the compounds of this invention are capable of forming acid and/or base salts by virtue of the presence of amino and/or carboxyl groups or groups similar thereto. Pharmaceutically acceptable base addition salts can be prepared from inorganic and organic bases. Salts derived from inorganic bases, include by way of example only, sodium, potassium, lithium, ammonium, calcium and magnesium salts. Salts derived from organic bases include, but are not limited to, salts of primary, secondary and tertiary amines, such as alkyl amines, dialkyl amines, trialkyl amines, substituted alkyl amines, di(substituted alkyl) amines, tri(substituted alkyl) amines, alkenyl amines, dialkenyl amines, trialkenyl amines, substituted alkenyl amines, di(substituted alkenyl) amines, tri(substituted alkenyl) amines, cycloalkyl amines, di(cycloalkyl) amines, tri(cycloalkyl) amines, substituted cycloalkyl amines, disubstituted cycloalkyl amines, trisubstituted cycloalkyl amines, cycloalkenyl amines, di(cycloalkenyl) amines, tri(cycloalkenyl) amines, substituted cycloalkenyl amines, disubstituted cycloalkenyl amines, trisubstituted cycloalkenyl amines, aryl amines, diaryl amines, triaryl amines, heteroaryl amines, diheteroaryl amines, triheteroaryl amines, heterocyclic amines, diheterocyclic amines, triheterocyclic amines, mixed di- and tri-amines where at least two of the substituents on the amine are different and are selected from the group consisting of alkyl, substituted alkyl, alkenyl, substituted alkenyl, cycloalkyl, substituted cycloalkyl, cycloalkenyl, substituted cycloalkenyl, aryl, heteroaryl, heterocyclic, and the like. Also included are amines where the two or three substituents, together with the amino nitrogen, form a heterocyclic or heteroaryl group.

Pharmaceutically acceptable acid addition salts may be prepared from inorganic and organic acids. The inorganic acids that can be used include hydrochloric acid, hydrobromic acid, sulfuric acid, nitric acid, phosphoric acid, and the like. The organic acids that can be used include acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluenesulfonic acid, salicylic acid, and the like.

Examples of such pharmaceutically acceptable salts are the iodide, acetate, phenyl acetate, trifluoroacetate, acryl ate, ascorbate, benzoate, chlorobenzoate, dinitrobenzoate, hydroxybenzoate, methoxybenzoate, methylbenzoate, o-acetoxybenzoate, naphthalene-2-benzoate, bromide, isobutyrate, phenylbutyrate, g-hydroxybutyrate, b-hydroxybutyrate, butyne-1,4-dioate, hexyne-1,4-dioate, hexyne-1,6-dioate, caproate, caprylate, chloride, cinnamate, citrate, decanoate, formate, fumarate, glycollate, heptanoate, hippurate, lactate, malate, maleate, hydroxymaleate, malonate, mandelate, mesylate, nicotinate, isonicotinate, nitrate, oxalate, phthalate, terephthalate, phosphate, monohydrogenphosphate, dihydrogenphosphate, metaphosphate, pyrophosphate, propiolate, propionate, phenylpropionate, salicylate, sebacate, succinate, suberate, sulfate, bisulfate, pyrosulfate, sulfite, bisulfite, sulfonate, benzenesulfonate, p-bromophenylsulfonate, chlorobenzenesulfonate, propanesulfonate, ethanesulfonate, 2-hydroxyethanesulfonate, methanesulfonate, naphthalene-1-sulfonate, naphthalene-2-sulfonate, p-toluenesulfonate, xylenesulfonate, tartarate, and the like of a compound of formula I.

By “pharmaceutically acceptable” it is also meant that in a formulation containing the compound of Formula I, the carrier, diluent, excipients, and salt must be compatible with the other ingredients of the formulation, and not deleterious to the recipient thereof.

“Texaphyrin” means an aromatic pentadentate macrocyclic expanded porphyrin, also described as an aromatic benzannulene containing both 18π- and 22π-electron delocalization pathways. Texaphyrins and water-soluble texaphyrins, methods of preparation and various uses have been described in U.S. Pat. Nos. 4,935,498, 5,162,509, 5,252,720, 5,256,399, 5,272,142, 5,292,414, 5,369,101, 5,432,171, 5,439,570, 5,451,576, 5,457,183, 5,475,104, 5,504,205, 5,525,325, 5,559,207, 5,565,552, 5,567,687, 5,569,759, 5,580,543, 5,583,220, 5,587,371, 5,587,463, 5,591,422, 5,594,136, 5,595,726, 5,599,923, 5,599,928, 5,601,802, 5,607,924, 5,622,946, and 5,714,328; PCT publications WO 90/10633, 94/29316, 95/10307, 95/21845, 96/09315, 96/40253, 96/38461, 97/26915, 97/35617, 97/46262, and 98/07733; allowed U.S. patent application Ser. Nos. 08/458,347, 08/591,318, and 08/914,272; and pending U.S. patent application Ser. Nos. 08/763,451, 08/903,099, 08/946,435, 08/975,090, 08/975,522, 08/988,336, and 08/975,526; each of which are herein incorporated by reference in their entirety.

Prodrugs are derivatives of the compounds of the invention that have metabolically cleavable groups and become by solvolysis or under physiological conditions the compounds of the invention that are pharmaceutically active in vivo. For example, ester derivatives of compounds of this invention are often active in vivo, but not in vitro. Other derivatives of the compounds of this invention have activity in both their acid and acid derivative forms, but the acid derivative form often offers advantages of solubility, tissue compatibility, or delayed release in the mammalian organism (see, Bundgard, H., Design of Prodrugs, pp. 7-9, 21-24, Elsevier, Amsterdam 1985). Prodrugs include acid derivatives well known to practitioners of the art, such as, for example, esters prepared by reaction of the parent acid with a suitable alcohol, or amides prepared by reaction of the parent acid compound with an amine. Simple aliphatic or aromatic esters derived from acidic groups pendant on the compounds of this invention are preferred prodrugs. In some cases it is desirable to prepare double ester type prodrugs such as (acyloxy)alkyl esters or ((alkoxycarbonyl)oxy)alkyl esters.

The term “therapeutically effective amount” refers to the amount of a compound of Formula I that is sufficient to effect treatment, as defined below, when administered to a mammal in need of such treatment. The therapeutically effective amount will vary depending upon the subject and disease condition being treated, the weight and age of the subject, the severity of the disease condition, the particular compound of Formula I chosen, the dosing regimen to be followed, timing of administration, the manner of administration and the like, all of which can readily be determined by one of ordinary skill in the art.

The term “treatment” or “treating” means any treatment of a disease in a mammal, including:
a) preventing the disease, that is, causing the clinical symptoms of the disease not to develop;
b) inhibiting the disease, that is, arresting the development of clinical symptoms; and/or
c) relieving the disease, that is, causing the regression of clinical symptoms.

1. A compound of Formula I: 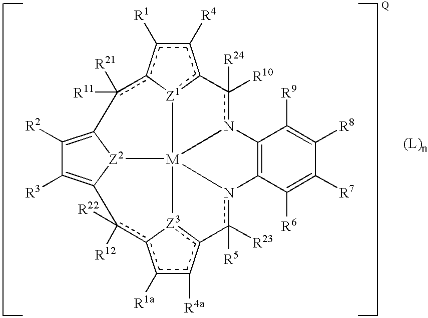
its hydrate or pharmaceutically acceptable salt thereof, wherein:
M represents H or a metal cation;
Q represents an integer of from about −5 to about +5;
L represents a charge balancing species; “n” represents an integer of from 0 to +5;
Z¹, Z² and Z³ independently represent N or O;
R¹, R^{1a}, R², R³, R⁴, R^{4a}, R⁷, and R⁸ are independently selected from acyl, acyloxy, optionally substituted alkenyl, optionally substituted alkoxy, optionally substituted alkyl, optionally substituted alkynyl, optionally substituted amino, optionally substituted aryl, optionally substituted aryloxy, carboxyl, (optionally substituted alkoxy)carbonyl, (optionally substituted amino)carbonyl, (optionally substituted alkoxy)carbonyloxy, (optionally substituted amino)carbonyloxy, cyano, optionally substituted cycloalkyl, optionally substituted cycloalkenyl, halogen, optionally substituted heteroaryl, optionally substituted heteroaryloxy, optionally substituted heterocyclyl, optionally substituted heterocyclooxy, hydrogen, hydroxyl, nitro, optionally substituted azo, S—R³¹, SO—R³¹ and SO₂—R³¹;
R⁶ and R⁹ are independently selected from acyl, acyloxy, optionally substituted alkenyl, optionally substituted alkoxy, optionally substituted alkyl, optionally substituted alkynyl, optionally substituted amino, optionally substituted aryl, optionally substituted aryloxy, carboxyl, (optionally substituted alkoxy)carbonyl, (optionally substituted amino)carbonyl, (optionally substituted alkoxy)carbonyloxy, (optionally substituted amino)carbonyloxy, cyano, optionally substituted cycloalkyl, optionally substituted cycloalkenyl, fluoro, chloro, bromo, optionally substituted heteroaryl, optionally substituted heteroaryloxy, optionally substituted heterocyclyl, optionally substituted heterocyclooxy, hydrogen, hydroxyl, nitro, optionally substituted azo, sulfanyl, sulfinyl and sulfonyl;
R⁵, R¹⁰, R¹¹ and R¹² are independently selected from acyl, optionally substituted alkoxy, optionally substituted alkyl, optionally substituted aryl, halo, hydrogen, hydroxy, optionally substituted cycloalkyl, optionally substituted cycloalkenyl, optionally substituted heteroaryl, and optionally substituted heterocyclyl;
R²¹, R²², R²³ and R²⁴ are optional substituents, and when present are independently selected from H, OH, optionally substituted C₁₋₄ alkyl, optionally substituted C₃₋₁₀ cycloalkyl, optionally substituted aryl, optionally substituted heteroaryl, optionally substituted heterocyclyl;
R³¹ represents acyl, optionally substituted alkenyl, optionally substituted alky, optionally substituted alkoxy, optionally substituted alkoxycarbonyl, optionally substituted alkynyl, optionally substituted aminocarbonyl, optionally substituted aryl, carboxy, optionally substituted cycloalkyl, optionally substituted heteroaryl, or optionally substituted heterocyclyl;
alternatively
R⁵ and R²³ along with the carbon atom to which they are attached represent C═O, C═S, C═CHR³¹ or C═N—R³¹;
R¹⁰ and R²⁴ along with the carbon atom to which they are attached represent C═O, C═S, C═CHR³¹ or C═N—R³¹;
R¹¹ and R²¹ along with the carbon atom to which they are attached represent C═O, C═S, C═CHR³¹ or C═N—R³¹;
R¹² and R²² along with the carbon atom to which they are attached represent C═O, C═S, C═CHR³¹ or C═N—R³¹;
R¹ and R²¹,
R¹¹ and R²,
R³ and R²²,
R¹² and R^{1a},
R^{4a} and R⁵,
R²³ and R⁶,
R⁹ and R²⁴, and
R¹⁰ and R⁴ respectively taken together represent W(CHR³²)₁₋₈ or (CHR³²)₁₋₈, wherein R³² represents H, methyl, or ethyl, and W represents O, NR³¹, or S, with the proviso that:
(a) at least one of
R⁵ and R²³,
R¹⁰ and R²⁴,
R¹¹ and R²¹, and
R¹² and R²² respectively taken together with the carbon atom to which they are attached represent C═O, C═S, C═CHR³¹ or C═N—R³¹; or
(b) at least one of
R¹ and R²¹,
R¹¹ and R²,
R³ and R²²,
R¹² and R^{1a},
R^{4a} and R⁵,
R²³ and R⁶,
R⁹ and R²⁴, and
R¹⁰ and R⁴ respectively, taken together represent W(CHR³²)₁₋₈ or (CHR³²)₁₋₈, where R³² represents H, methyl or ethyl.

2. A Formula I compound of claim 1: 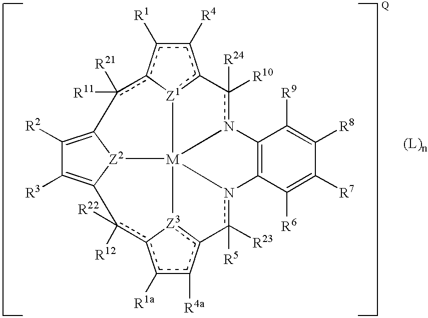
its hydrate, pharmaceutically acceptable salt or prodrug form thereof, wherein:
M represents H or a metal cation;
Q represents an integer of from −4 to +4;
L represents a charge-balancing group;
“n” represents an integer of from about 0 to about +4;
Z¹, Z² and Z³ independently represent N or O;
R¹, R^{1a}, R², R³, R⁴, R^{4a}, R⁷, and R⁸ are independently selected from acyl, acyloxy, optionally substituted alkenyl, optionally substituted alkoxy, optionally substituted alkyl, optionally substituted alkynyl, optionally substituted amino, optionally substituted aryl, optionally substituted aryloxy, carboxyl, (optionally substituted alkoxy)carbonyl, (optionally substituted amino)carbonyl, (optionally substituted alkoxy)carbonyloxy, (optionally substituted amino)carbonyloxy, cyano, optionally substituted cycloalkyl, optionally substituted cycloalkenyl, halogen, optionally substituted heteroaryl, optionally substituted heteroaryloxy, optionally substituted heterocyclyl, optionally substituted heterocyclooxy, hydrogen, hydroxyl, nitro, optionally substituted azo, sulfanyl, sulfinyl and sulfonyl;
R⁶ and R⁹ are independently selected from acyl, acyloxy, optionally substituted alkenyl, optionally substituted alkoxy, optionally substituted alkyl, optionally substituted alkynyl, optionally substituted amino, optionally substituted aryl, optionally substituted aryloxy, carboxyl, (optionally substituted alkoxy)carbonyl, (optionally substituted amino)carbonyl, (optionally substituted alkoxy)carbonyloxy, (optionally substituted amino)carbonyloxy, cyano, optionally substituted cycloalkyl, optionally substituted cycloalkenyl, fluoro, chloro, bromo, optionally substituted heteroaryl, optionally substituted heteroaryloxy, optionally substituted heterocyclyl, optionally substituted heterocyclooxy, hydrogen, hydroxyl, nitro, optionally substituted azo, sulfanyl, sulfinyl and sulfonyl;
R⁵, R¹⁰, R¹¹ and R¹² are independently selected from acyl, optionally substituted alkoxy, optionally substituted alkyl, optionally substituted aryl, halo, hydrogen, hydroxy optionally substituted cycloalkyl, optionally substituted cycloalkenyl, optionally substituted heteroaryl, optionally substituted heterocyclyl, and OH;
R²¹, R²², R²³ and R²⁴ are optional substituents, and when present are independently selected from H, OH, C₁₋₄ alkyl, C₃₋₁₀ cycloalkyl, aryl, heteroaryl, and heterocyclyl;
alternatively
R¹ and R²¹,
R¹¹ and R²,
R³ and R²²,
R¹² and R^{1a},
R^{4a} and R⁵,
R²³ and R⁶,
R⁹ and R²⁴, and
R¹⁰ and R⁴ respectively, taken together represent W(CHR³²)₁₋₈ or (CHR³²)₁₋₈, wherein R³² represents H, methyl, or ethyl, and W represents O, NR³, or S;
with the proviso that
at least one of
R¹ and R²¹,
R¹¹ and R²,
R³ and R²²,
R¹² and R^{1a},
R^{4a} and R⁵,
R^{4a} and R⁵,
R²³ and R⁶,
R⁹ and R²⁴, and
R¹⁰ and R⁴ respectively taken together represent W(CHR³²)₁₋₈ or (CHR³²)₁₋₈.

3. A Formula I compound of claim 1: 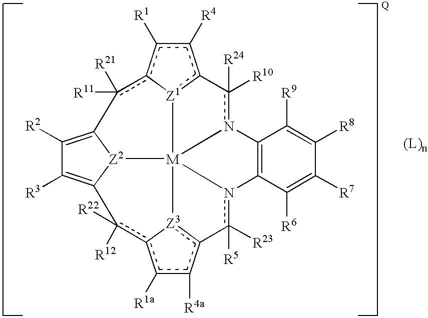
its hydrate, pharmaceutically acceptable salt or prodrug form thereof, wherein:
M represents H or a metal cation;
Q represents an integer of from −5 to +5;
L represents a charge balancing species; “n” represents an integer of from 0 to +5;
Z¹, Z² and Z³ independently represent N or O;
R¹, R^{1a}, R², R³, R⁴, R^{4a}, R⁷, and R⁸ are independently selected from acyl, acyloxy, optionally substituted alkenyl, optionally substituted alkoxy, optionally substituted alkyl, optionally substituted alkynyl, optionally substituted amino, optionally substituted aryl, optionally substituted aryloxy, carboxyl, (optionally substituted alkoxy)carbonyl, (optionally substituted amino)carbonyl, (optionally substituted alkoxy)carbonyloxy, (optionally substituted amino)carbonyloxy, cyano, optionally substituted cycloalkyl, optionally substituted cycloalkenyl, halogen, optionally substituted heteroaryl, optionally substituted heteroaryloxy, optionally substituted heterocyclyl, optionally substituted heterocyclooxy, hydrogen, hydroxyl, nitro, optionally substituted azo, S—R³¹, SO—R³¹ and SO₂—R³¹;
R⁶ and R⁹ are independently selected from acyl, acyloxy, optionally substituted alkenyl, optionally substituted alkoxy, optionally substituted alkyl, optionally substituted alkynyl, optionally substituted amino, optionally substituted aryl, optionally substituted aryloxy, carboxyl, (optionally substituted alkoxy)carbonyl, (optionally substituted amino)carbonyl, (optionally substituted alkoxy)carbonyloxy, (optionally substituted amino)carbonyloxy, cyano, optionally substituted cycloalkyl, optionally substituted cycloalkenyl, fluoro, chloro, bromo, optionally substituted heteroaryl, optionally substituted heteroaryloxy, optionally substituted heterocyclyl, optionally substituted heterocyclooxy, hydrogen, hydroxyl, nitro, optionally substituted azo, sulfanyl, sulfinyl and sulfonyl;
R⁵, R¹⁰, R¹¹ and R¹² are independently selected from acyl, optionally substituted alkoxy, optionally substituted alkyl, optionally substituted aryl, halo, hydrogen, hydroxy optionally substituted cycloalkyl, optionally substituted cycloalkenyl, optionally substituted heteroaryl, and optionally substituted heterocyclyl;
R²¹, R²², R²³ and R²⁴ are optional substituents, and when present are independently selected from H, OH, optionally substituted C₁₋₄ alkyl, optionally substituted C₃₋₁₀ alkyl, optionally substituted aryl, optionally substituted heteroaryl, optionally substituted heterocyclyl;
alternatively
R⁵ and R²³ along with the carbon atom to which they are attached represent C═O, C═S, C═CHR³¹ or C═N—R³¹;
R¹⁰ and R²⁴ along with the carbon atom to which they are attached represent C═O, C═S, C═CHR³¹ or C═N—R³¹;
R¹¹ and R²¹ along with the carbon atom to which they are attached represent C═O, C═S, C═CHR³¹ or C═N—R³¹;
R¹² and R²² along with the carbon atom to which they are attached represent C═O, C═S, C═CHR³¹ or C═N—R³¹;
R³¹ represents acyl, optionally substituted alkenyl, optionally substituted alky, optionally substituted alkoxy, optionally substituted alkoxycarbonyl, optionally substituted alkynyl, optionally substituted aminocarbonyl, optionally substituted aryl, carboxy, optionally substituted cycloalkyl, optionally substituted heteroaryl, or optionally substituted heterocyclyl;
with the proviso that:
at least one of
R⁵ and R²³,
R¹⁰ and R²⁴,
R¹¹ and R²¹, and
R¹² and R²²,
along with the respective carbon atoms to which they are attached represent C═O, C═S, C═CHR³¹ or C═N—R³¹.

4. A compound of claim 3 wherein
M is selected from H, and a divalent or trivalent metal cation;
Z¹ and Z² independently represent N or O;
Z³ represents N;
R¹, R^{1a}, R², R³, R⁴, R^{4a}, R⁷, and R⁸ are independently selected from acyl, acyloxy, optionally substituted alkoxy, optionally substituted alkyl, optionally substituted amino, optionally substituted aryl, optionally substituted aryloxy, carboxyl, (optionally substituted amino)carbonyl, cyano, optionally substituted cycloalkyl, optionally substituted cycloalkenyl, halogen, optionally substituted heteroaryl, optionally substituted heteroaryloxy, optionally substituted heterocyclyl, hydrogen, hydroxyl, nitro, optionally substituted azo, S—R³¹, SO—R³¹, and SO₂—R³¹;
R⁶ and R⁹ are independently selected from acyl, acyloxy, optionally substituted alkoxy, optionally substituted alkyl, optionally substituted amino, optionally substituted aryl, optionally substituted aryloxy, carboxyl, (optionally substituted amino)carbonyl, cyano, optionally substituted cycloalkyl, optionally substituted cycloalkenyl, halogen, optionally substituted heteroaryl, optionally substituted heteroaryloxy, optionally substituted heterocyclyl, hydrogen, hydroxyl, nitro, optionally substituted azo, S—R³¹, SO—R³¹, and SO₂—R³¹;
R²¹, R²², R²³ and R²⁴ are optional substituents, and when present are independently selected from H, optionally substituted C₁₋₄ alkyl, optionally substituted C₃₋₁₀ cyclo alkyl, optionally substituted aryl, optionally substituted heteroaryl, optionally substituted heterocyclyl;
alternatively
R⁵ and R²³ along with the carbon atom to which they are attached represent C═O, C═S, or C═N—R³¹;
R¹⁰ and R²⁴ along with the carbon atom to which they are attached represent C═O, C═S, or C═N—R³¹;
R¹¹ and R²¹ along with the carbon atom to which they are attached represent C═O, C═S, or C═N—R³¹;
R¹² and R²² along with the carbon atom to which they are attached represent C═O, C═S, or C═N—R³¹;
R³¹ represents acyl, optionally substituted alky, optionally substituted alkoxy, optionally substituted aminocarbonyl, optionally substituted aryl, carboxy, optionally substituted cycloalkyl, optionally substituted heteroaryl, or optionally substituted heterocyclyl; with the proviso that at least one of
R⁵ and R²³,
R¹⁰ and R²⁴,
R¹¹ and R²¹, and
R¹² and R²² along with the respective carbon atoms to which they are attached represent C═O or C═N—R³¹.

5. A compound of claim 4 wherein
M represents H or a metal cation selected from Lu(III), Fe(II), Fe(III), Ni(II), Cd(II), In(III), Gd (III), Sm(III), Eu(III), Tb(III), Dy(III), Er(III), Tm(III), Yb(III), Mn(II), Y(III), and Co (II); and
L represents a charged species selected from H⁺, NH₄₊, Na⁺, Ca²⁺, K⁺, Ba²⁺, Cl⁻, Br⁻, SO₄²⁻, carboxylate (CH₃COO⁻, C₂H₅COO⁻), sulfonate (RSO₃⁻), phosphate, cholate, gluconate, glucuronate, and citrate.

6. A compound of claim 5 wherein:
M represents Lu(III), Gd (III), Er(III), Tb(III), Dy(III), Ho(III), Y(III), Tm(III), Yb(III), or Mn(II);
R¹, R^{1a}, R², R³, R⁴, R^{4a}, R⁷, and R⁸ are independently selected from C₁₋₅ alkyl, C₁₋₅ hydroxyalkyl, C₁₋₅ alkoxyalkyl, C₁₋₅ carboxyalkyl, acetoxyalkyl, aryloxyalkyl, R(CH₂CH₂O)ₙ, where n=1-5, and R═C₁₋₂ alkyl, phenyl, hydroxyl, carboxyl;
R⁶ and R⁹ are independently selected from acyl, acyloxy, optionally substituted alkoxy, optionally substituted alkyl, optionally substituted amino, optionally substituted aryl, optionally substituted aryloxy, carboxyl, (optionally substituted alkoxy)carbonyl, (optionally substituted amino)carbonyl, (optionally substituted amino)carbonyloxy, cyano, fluoro, chloro, hydrogen, hydroxyl, and nitro;
R⁵, R¹⁰, R¹¹ and R¹² are independently selected from H, OH, C₁₋₂ alkyl, C₁₋₃ acyl, and phenyl;
R²¹, R²², R²³ and R²⁴ are optional substituents, and when present are independently selected from H, OH, optionally substituted C₁₋₄ alkyl, optionally substituted C₃₋₁₀ alkyl, optionally substituted aryl, optionally substituted heteroaryl, optionally substituted heterocyclyl;
alternatively
R⁵ and R²³ along with the carbon atom to which they are attached represent C═O or C═N—R³¹;
R¹⁰ and R²⁴ along with the carbon atom to which they are attached represent C═O or C═N—R³¹;
R¹¹ and R²¹ along with the carbon atom to which they are attached represent C═O or C═N—R³¹;
R¹² and R²² along with the carbon atom to which they are attached represent C═O or C═N—R³¹;
R³¹ represents acyl, optionally substituted alky, optionally substituted alkoxy, optionally substituted alkoxycarbonyl, optionally substituted aminocarbonyl, optionally substituted aryl, or carboxy;
with the proviso that at least one of
R⁵ and R²³,
R¹⁰ and R²⁴,
R¹¹ and R²¹, and
R¹² and R²² along with the respective carbon atoms to which they are attached represent C═O.

7. A compound of claim 6 wherein:
M represents Lu(III), Gd(III), Tb(III), Mn(II), Co(II), Y(III), or Ho(III);
Z¹, Z² and Z³ independently represent N.

8. A compound of Formula I 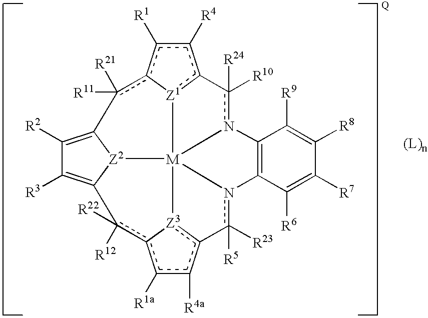
its hydrate, pharmaceutically acceptable salt, or prodrug form thereof, wherein:
M represents Lu(III), Gd(III), Tb(III), Mn(II), Co(II), Y(III), or Ho(III);
Z¹, Z² and Z³ independently represent N or O;
R¹, R^{1a}, R², R³, R⁴, R^{4a}, R⁷, and R⁵ are independently selected from C₁₋₅ alkyl, C₁₋₅ hydroxyalkyl, C₁₋₅ alkoxyalkyl, C₁₋₅ carboxyalkyl, acetoxyalkyl, aryloxyalkyl, R(CH₂CH₂O)ₙ, where n=1-5, and R═C₁₋₂ alkyl, phenyl, hydroxyl, carboxyl;
R⁶ and R⁹ are independently selected from acyl, acyloxy, optionally substituted alkoxy, optionally substituted alkyl, optionally substituted amino, optionally substituted aryl, optionally substituted aryloxy, carboxyl, (optionally substituted alkoxy)carbonyl, (optionally substituted amino)carbonyl, (optionally substituted amino)carbonyloxy, cyano, fluoro, chloro, hydrogen, hydroxyl, and nitro;
R⁵, R¹⁰, R¹¹ and R¹² are independently selected from H, OH, C₁₋₂ alkyl, C₁₋₃ acyl, and phenyl;
R²¹, R²², R²³ and R²⁴ are optional substituents, and when present are independently selected from H, OH, optionally substituted C₁₋₄ alkyl, optionally substituted C₃₋₁₀ alkyl, optionally substituted aryl, optionally substituted heteroaryl, optionally substituted heterocyclyl;
alternatively
R⁵ and R²³ along with the carbon atom to which they are attached represent C═O or C═N—R³¹;
R¹⁰ and R²⁴ along with the carbon atom to which they are attached represent C═O or C═N—R³¹;
R¹¹ and R²¹ along with the carbon atom to which they are attached represent C═O or C═N—R³¹ R¹² and R²² along with the carbon atom to which they are attached represent C═O or C═N—R³¹;
R³¹ represents acyl, optionally substituted alky, optionally substituted alkoxy, optionally substituted alkoxycarbonyl, optionally substituted aminocarbonyl, optionally substituted aryl, or carboxy;
with the proviso that at least one of
R⁵ and R²³,
R¹⁰ and R²⁴,
R¹¹ and R²¹, and
R¹² and R²² along with the respective carbon atoms to which they are attached represent C═O.

9. A compound of Formula I of claim 8: 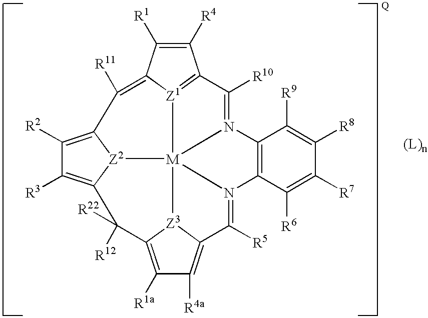
its hydrate, pharmaceutically acceptable salt, or prodrug form thereof, wherein:
M represents Lu(III) or Gd(III);
L represents OAc;
“n” represents the integer 2;
Z¹, Z² and Z³ represent N;
R¹ and R^{1a} represent (CH₂)₃OH;
R² represents C₂H₅;
R represents C₂H₅;
R⁴ and R^{4a} CH₃;
R⁵ represents H;
R⁶ represents CH₃;
R⁷ and R⁸ are independently selected from H and O—(CH₂—CH₂—O)₃—CH₃;
R⁹ represents CH₃;
R¹⁰ and R¹¹ represent H; and
R¹² and R²² along with the carbon atom to which they are attached represent C═O.

10. A pharmaceutical composition, comprising a pharmaceutically acceptable carrier and a therapeutically effective amount of a compound of claim 3 or a pharmaceutically acceptable salt form thereof.

11. A pharmaceutical composition, comprising a pharmaceutically acceptable carrier and a therapeutically effective amount of a compound of claim 8 or a pharmaceutically acceptable salt form thereof.

12. A method of treating a host harboring a neoplasm or atheroma, comprising administering to the host a compound of Formula I of claim 3, and administering ionizing radiation to the host in proximity to the neoplasm or atheroma.

13. A method of treating a host harboring a neoplasm or atheroma, comprising administering to the host a compound of Formula I of claim 8, and administering ionizing radiation to the host in proximity to the neoplasm or atheroma.

14. A compound of claim 2 wherein,
M is selected from H, a divalent metal cation and a trivalent metal cation;
R¹, R^{1a}, R², R³, R⁴, R^{4a}, R⁷, and R⁸ are independently selected from acyl, acyloxy, optionally substituted alkoxy, optionally substituted alkyl, optionally substituted amino, optionally substituted aryl, optionally substituted aryloxy, carboxyl, cycloalkyl, optionally substituted cycloalkenyl, halogen, optionally substituted heteroaryl, optionally substituted heteroaryloxy, optionally substituted heterocyclyl, hydrogen, hydroxyl, nitro, optionally substituted azo, S—R³¹, SO—R³¹, and SO₂—R³¹;
R⁶ and R⁹ are independently selected from acyl, acyloxy, optionally substituted alkoxy, optionally substituted alkyl, optionally substituted amino, optionally substituted aryl, optionally substituted aryloxy, carboxyl, cycloalkyl, cycloalkenyl, halogen, optionally substituted heteroaryl, optionally substituted heteroaryloxy, optionally substituted heterocyclyl, hydrogen, hydroxyl, nitro, optionally substituted azo, S—R³¹, and SO₂—R³¹;
R²¹, R²², R²³ and R²⁴ are optional substituents, and when present are independently selected from H, C₁₋₄ alkyl, C₃₋₁₀ cyclo alkyl, optionally substituted aryl, five to ten membered heteroaryl, optionally substituted heterocyclyl;
C₃₋₁₀ cyclo alkyl substituted with halogen, O(CH₂)₀₋₂—CH₃,
C₁₋₄ alkyl substituted with halogen, O(CH₂)₀₋₂—CH₃, and
five to ten membered heteroaryl substituted with halogen, C₁₋₄ alkyl, O(CH₂)₀₋₂—CH₃;
alternatively
R³¹ represents acyl, optionally substituted alky, optionally substituted alkoxy, optionally substituted aminocarbonyl, optionally substituted aryl, carboxy, optionally substituted cycloalkyl, optionally substituted heteroaryl, or optionally substituted heterocyclyl;
alternatively, one or more of
R¹ and R²¹,
R¹¹ and R²,
R³ and R²²,
R¹² and R^{1a},
R^{4a} and R⁵,
R²³ and R⁶,
R⁹ and R²⁴, and
R¹⁰ and R⁴ respectively taken together represent —O(CHR³¹)₁₄— or —(CHR³¹)₁₋₄—, wherein R³¹ represents H, methyl, or ethyl;
with the proviso that
at least one of
R¹ and R²′,
R¹¹ and R²,
R³ and R²²,
R¹² and R^{1a},
R^{4a} and R⁵,
R²³ and R⁶,
R⁹ and R²⁴, and
R¹⁰ and R⁴ respectively taken together represent O(CHR³²)₁₋₄- or (CHR³²)₁₋₄-, wherein R³² represents H, methyl, or ethyl.

15. A compound of claim 14 wherein:
M represents H or a metal cation selected from Lu(III), Fe(II), Fe(III), Ni(II), Cd(II), In(III), Gd (III), Sm(III), Eu(III), Tb(III), Dy(III), Er(III), Tm(III), Yb(III), Mn(II), Y(III), and Co (II); and
L represents a charged species selected from H⁺, NH₄⁺, Na⁺, Ca⁺⁺, K⁺, Ba⁺⁺, Cl⁻, Br⁻,
SO₄²⁻, carboxylate (CH₃COO⁻, C₂H₅COO⁻), sulfonate (RSO₃—), phosphate, cholate, gluconate, glucuronate, and citrate.

16. A compound of claim 15 wherein:
M represents Lu(III), Gd (III), Er(III), Tb(III), Dy(III), Ho(III), Y(III), Tm(III), Yb(III), or Mn(II);
Z¹, Z² and Z³ represent N;
R¹, R^{1a}, R², R³, R⁴, R^{4a}, R⁷, and R⁸ are independently selected from C₁₋₅ alkyl, C₁₋₅ hydroxyalkyl, C₁₋₅ alkoxyalkyl, C₁₋₅ carboxyalkyl, acetoxyalkyl, aryloxyalkyl, R(CH₂CH₂O)ₙ, where n=1-5, and R═C₁₋₂ alkyl, phenyl, hydroxyl, carboxyl;
R⁶ and R⁹ are independently selected from acyl, acyloxy, optionally substituted alkoxy, optionally substituted alkyl, optionally substituted amino, optionally substituted aryl, optionally substituted aryloxy, carboxyl, (optionally substituted alkoxy)carbonyl, (optionally substituted amino)carbonyl, (optionally substituted amino)carbonyloxy, cyano, fluoro, chloro, hydrogen, hydroxyl, and nitro;
R⁵, R¹⁰, R¹¹ and R¹² are independently selected from H, OH, C₁₋₂ alkyl, C₁₋₃ acyl, and phenyl;
R²¹, R²², R²³ and R²⁴ are optional substituents, and when present are independently selected from H, OH, optionally substituted C₁₋₄ alkyl, optionally substituted C₃₋₁₀ alkyl, optionally substituted aryl, optionally substituted heteroaryl, optionally substituted heterocyclyl;
alternatively, one or more of
R¹ and R²¹,
R¹¹ and R²,
R³ and R²²,
R¹² and R^{1a},
R^{4a} and R⁵,
R²³ and R⁶,
R⁹ and R²⁴, and
R¹⁰ and R⁴ respectively taken together represent O(CHR³²)₁₋₄ or (CHR³²)₁₋₄, wherein R³² represents H, methyl, or ethyl;
with the proviso that
at least one of
R¹ and R²¹,
R¹¹ and R²,
R³ and R²²,
R¹² and R^{1a},
R^{4a} and R⁵,
R²³ and R⁶,
R⁹ and R²⁴, and
R¹⁰ and R⁴ respectively taken together represent O(CHR³²)₂₋₄ or (CHR³²)₂₋₄, wherein R³² represents H, methyl, or ethyl.

17. A compound of claim 16, wherein
M represents Lu(III), Gd(III), Tb(III), Mn(II), Co(II), Y(III), or Ho(III).

18. A compound of Formula I: 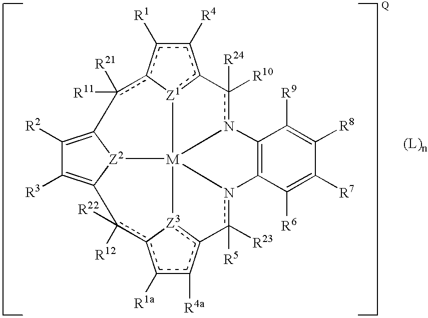
its hydrate, pharmaceutically acceptable salt or prodrug form thereof, wherein:
M represents Lu(III), Gd(III), Tb(III), Mn(II), Co(II), Y(III), or Ho(III);
Z¹, Z² and Z³ independently represent N;
R¹, R^{1a}, R², R³, R⁴, R^{4a}, R⁷, and R⁸ are independently selected from C₁₋₅ alkyl, C₁₋₅ hydroxyalkyl, C₁₋₅ alkoxyalkyl, C₁₋₅ carboxyalkyl, acetoxyalkyl, aryloxyalkyl, R(CH₂CH₂O)ₙ, where n=1-5, and R═C₁₋₂ alkyl, phenyl, hydroxyl, carboxyl;
R⁶ and R⁹ are independently selected from acyl, acyloxy, optionally substituted alkoxy, optionally substituted alkyl, optionally substituted amino, optionally substituted aryl, optionally substituted aryloxy, carboxyl, (optionally substituted alkoxy)carbonyl, (optionally substituted amino)carbonyl, (optionally substituted amino)carbonyloxy, cyano, fluoro, chloro, hydrogen, hydroxyl, and nitro;
R⁵, R¹⁰, R¹¹ and R¹² are independently selected from H, OH, C₁₋₂ alkyl, C₁₋₃ acyl, and phenyl;
R²¹, R²², R²³ and R²⁴ are optional substituents, and when present are independently selected from H, OH, optionally substituted C₁₋₄ alkyl, optionally substituted C₃₋₁₀ alkyl, optionally substituted aryl, optionally substituted heteroaryl, optionally substituted heterocyclyl;
alternatively, at least one of
R¹ and R²¹,
R¹¹ and R²,
R³ and R²²,
R¹² and R^{1a},
R^{4a} and R⁵,
R²³ and R⁶,
R⁹ and R²⁴, and
R¹⁰ and R⁴ respectively taken together represent O(CHR³²)₁₋₄ or (CHR³²)₁₋₄, wherein R³² represents H, methyl, or ethyl;
with the proviso that at least one of
R¹ and R²¹,
R¹¹ and R²,
R³ and R²²,
R¹² and R^{1a},
R^{4a} and R⁵,
R²³ and R⁶,
R⁹ and R²⁴, and
R¹⁰ and R⁴ respectively taken together represent O(CHR³²)₂₋₄ or (CHR³²)₂₋₄, wherein R³² represents H, methyl, or ethyl.

19. A compound of Formula I of claim 18: 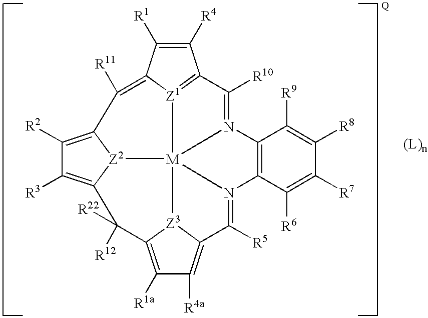
its hydrate, pharmaceutically acceptable salt or prodrug form thereof, wherein:
M represents Lu(III) or Gd(III);
Z¹, Z² and Z³ independently represent N;
L represents OAc;
“n” represents an integer of 2;
R¹ represents (CH₂)₃OH;
R² and R³ represent CH₃;
R⁴ and R^{4a} represent CH₃;
R⁵ and R⁶ represent H;
R⁷, and R⁸ are independently selected from O—(CH₂)₃—OH, OCH₃ and H;
R⁹ represents H; and
R¹² and R^{1a} taken together represent O(CHR³²)₃ wherein R³² represents H.

20. A pharmaceutical composition, comprising a pharmaceutically acceptable carrier and a therapeutically effective amount of a compound of claim 2 or a pharmaceutically acceptable salt form thereof.

21. A pharmaceutical composition, comprising a pharmaceutically acceptable carrier and a therapeutically effective amount of a compound of claim 17 or a pharmaceutically acceptable salt form thereof.

22. A method of treating a host harboring a neoplasm or atheroma, comprising administering to the host a compound of Formula I of claim 2, and administering ionizing radiation to the host in proximity to the neoplasm or atheroma.

23. A method of treating a host harboring a neoplasm or atheroma, comprising administering to the host a compound of Formula I of claim 17, and administering ionizing radiation to the host in proximity to the neoplasm or atheroma.
